(19) United States
(12) Patent Application Publication
(10) Pub. No.: US 2026/0158149 A1
(43) Pub. Date: Jun. 11, 2026

(54) GLYCOSYLATED PRODRUGS

(71) Applicant: UNIVERSITEIT LEIDEN, Leiden (NL)

(72) Inventors: Marcel SCHAAF, Leiden (NL); Annemarie MEIJER, Leiden (NL); Mahmoud HALIMA, Leiden (NL); Mei WANG, Leiden (NL); Hans AERTS, Leiden (NL); Nathaniel MARTIN, Leiden (NL); Karol AYED, Leiden (NL)

(73) Assignee: UNIVERSITEIT LEIDEN, Leiden (NL)

(21) Appl. No.: 18/706,583

(22) PCT Filed: Nov. 2, 2022
(86) PCT No.: PCT/NL2022/050621

§ 371 Date: May 1, 2024

(30) Foreign Application Priority Data
Nov. 2, 2021 (NL) 2029599

(51) Int. Cl. A61K 47/54; A61P 29/00

(52) U.S. Cl. CPC A61K 47/549; A61P 29/00

(57) ABSTRACT

Provided herein are compounds of formula A-B, where A is selected from a monosaccharide or a disaccharide and B is a therapeutic moiety (e.g. selected from an anti-inflammatory moiety or chemotherapeutic moiety); as well as a pharmaceutically acceptable salt, stereoisomer, acid, or solvate thereof. Such a compound may be a substrate for glucosylceramidase beta 2 (GBA2), such that the compound is a prodrug that provides an active compound after cleavage of the monosaccharide or disaccharide by GBA2. Also provided are pharmaceutical compositions comprising the compounds, as well as various medical and non-medical uses of the compounds and compositions.

### CROSS-REFERENCE TO RELATED APPLICATIONS

This application is a 35 U.S.C. § 371 National Phase Entry Application of International Application No. PCT/NL2022/050621 filed Nov. 2, 2022, which claims benefit under 35 U.S.C. § 119(a) of NL Application No. 2029599 filed Nov. 2, 2021, the contents of which are incorporated herein by reference in their entireties.

This invention relates to compounds comprising a monosaccharide or a disaccharide and a therapeutic moiety (such as an anti-inflammatory moiety or chemotherapeutic moiety), as well as a pharmaceutically acceptable salt, stereoisomer, acid, or solvate thereof. The compounds may be a substrate for glucosylceramidase beta 2 (GBA2), such that said compound is a prodrug that provides an active compound after cleavage of the monosaccharide or disaccharide by GBA2. Also provided are pharmaceutical compositions comprising the compounds, as well as various medical and non-medical uses of the compounds and compositions.

### BACKGROUND

Corticosteroids, such as glucocorticoids, are a class of compounds that are commonly used clinically because of their immune-suppressive action, such as prednisolone and dexamethasone (Burns, 2016, The History of Cortisone Discovery and Development. Rheum Dis Clin North Am 42, 1-14, vii). They are used in the treatment of a wide range of inflammatory and immune disorders such as asthma, rheumatoid arthritis, inflammatory bowel disease and psoriasis, hematological malignancies such as leukemia, and to alleviate inflammatory complications of infectious diseases such as tuberculosis and COVID-19 (Barnes, 2017, Glucocorticosteroids. Handb Exp Pharmacol 237, 93-115; Buttgereit, 2020, Views on glucocorticoid therapy in rheumatology: the age of convergence. Nat Rev Rheumatol 16, 239-246; Rhen and Cidlowski, 2005, Antiinflammatory action of glucocorticoids—new mechanisms for old drugs. N Engl J Med 353, 1711-1723). Although they are highly effective in attenuating inflammatory and other immune responses, their clinical use is limited by the severity of their side effects, which include an increased risk of diabetes, disruption of the endocrine system, osteoporosis, delayed and disrupted wound healing and inhibition of growth and fertility (Hoes et al., 2009, Adverse events of low- to medium-dose oral glucocorticoids in inflammatory diseases: a meta-analysis. Ann Rheum Dis 68, 1833-1838; McDonough et al., 2008, The epidemiology of glucocorticoid-associated adverse events. Curr Opin Rheumatol 20, 131-137; Schacke et al., 2002, Dissociation of transactivation from transrepression by a selective glucocorticoid receptor agonist leads to separation of therapeutic effects from side effects. Proc Natl Acad Sci USA 101, 227-232).

All effects of glucocorticoids are mediated by an intracellular receptor, the glucocorticoid receptor (GR), which is expressed in virtually all cells of our body (Nicolaides et al., 2010). Upon activation by a ligand, the cytoplasmic GR is activated and translocates to the nucleus, where it acts as a transcription factor, regulating the transcription of a large variety of target genes. The GR regulates gene expression in several ways (reviewed in (Meijsing, 2015, Mechanisms of Glucocorticoid-Regulated Gene Transcription. Adv Exp Med Biol 872, 59-81; Miranda et al., 2013, Complex genomic interactions in the dynamic regulation of transcription by the glucocorticoid receptor. Mol Cell Endocrinol 380, 16-24; Ramamoorthy and Cidlowski, 2016, Corticosteroids: Mechanisms of Action in Health and Disease. Rheum Dis Clin North Am 42, 15-31, vii; Ratman et al., 2013, Beck, I. M., and De Bosscher, K. (2013). How glucocorticoid receptors modulate the activity of other transcription factors: a scope beyond tethering. Mol Cell Endocrinol 380, 41-54). It can bind to specific sequences in the genome, so-called glucocorticoid-responsive elements (GREs), as a dimer (although binding as a tetramer has recently been described as well) and enhance or suppress transcription of a gene by recruiting specific transcriptional coregulator proteins which modulate the local chromatin structure and subsequent recruitment and activation of the transcription machinery. This process is generally called transactivation, although binding to a specific class of GREs (so-called negative GREs) may result in transcriptional suppression. Alternatively, the GR can, without dimerization, regulate gene transcription through interaction with other transcription factors. For example, it can physically interact with the p65 subunit of the NF-κB transcription factor complex and thereby inhibit the activity of this transcription factor. Generally, this process is called transrepression, even though some interactions between GR and other transcription factors may result in an enhanced transcriptional activity.

Initially, this transrepression activity of GR was considered the main mechanism underlying the anti-inflammatory actions of glucocorticoids, whereas most side effects of glucocorticoids were thought to result from the transactivation activity of GR (Schacke et al., 2002, Mechanisms involved in the side effects of glucocorticoids. Pharmacol Ther 96, 23-43; Schacke et al., 2004, Dissociation of transactivation from transrepression by a selective glucocorticoid receptor agonist leads to separation of therapeutic effects from side effects. Proc Natl Acad Sci USA 101, 227-232). Therefore, a lot of effort was put in developing novel GR agonists that induce the transrepression activity of GR but not its transactivation activity, in order to improve the therapeutic ratio of glucocorticoid drugs (Sundahl et al., 2015, Selective glucocorticoid receptor modulation: New directions with non-steroidal scaffolds. Pharmacol Ther 152, 28-41). However, although several promising compounds have been developed (see, for example, Baiula et al., 2014, Mapracorat, a selective glucocorticoid receptor agonist, causes apoptosis of eosinophils infiltrating the conjunctiva in late-phase experimental ocular allergy. Drug Des Devel Ther 8, 745-757; Biggadike et al., 2007, Nonsteroidal glucocorticoid agonists: tetrahydronaphthalenes with alternative steroidal A-ring mimetics possessing dissociated (transrepression/transactivation) efficacy selectivity. J Med Chem 50, 6519-6534; Dewint et al., 2008, A plant-derived ligand favoring monomeric glucocorticoid receptor conformation with impaired transactivation potential attenuates collagen-induced arthritis. J Immunol 180, 2608-2615; Stock et al., 2017, Improved disease activity with fosdagrocorat (PF-04171327), a partial agonist of the glucocorticoid receptor, in patients with rheumatoid arthritis: a Phase 2 randomized study. Int J Rheum Dis 20, 960-970; Zhang et al., 2020, Natural and synthetic compounds as dissociated agonists of glucocorticoid receptor. Pharmacol Res 156, 104802), success has been limited, most likely because the immune suppression by glucocorticoids it also may depend on the transactivation of anti-inflammatory genes and because some side effects also result from the transrepression activity (Clark, 2007, Anti-inflammatory functions of glucocorticoid-induced genes. Mol Cell Endocrinol 275, 79-97; Smoak and Cidlowski, 2004, Mechanisms of glucocorticoid receptor signaling during inflammation. Mech Ageing Dev 125, 697-706; Vandewalle et al., 2018, Therapeutic Mechanisms of Glucocorticoids. Trends Endocrinol Metab 29, 42-54). Therefore, it will be interesting to attempt novel approaches to develop glucocorticoid drugs with reduced side effects, such as the modification of the structure of existing GCs. For example, anti-CD163-dexamethasone conjugate, designed to target activated macrophages, showed an improved therapeutic ratio in rats (Thomsen et al., 2016, Anti-CD163-dexamethasone conjugate inhibits the acute phase response to lipopolysaccharide in rats. World J Hepatol 8, 726-730), and the conjugation of hydrolysable polyethylene glycol (PEG) to prednisolone increased the retention time in the lungs of rats, thereby not causing any systemic side effect (Bayard et al., 2013, Polyethylene glycol-drug ester conjugates for prolonged retention of small inhaled drugs in the lung. J Control Release 171, 234-240). Furthermore, the addition of γ-lactones and cyclic carbonates can make glucocorticoids more easily inactivatable by specific enzymes once they enter the blood stream (Biggadike et al., 2000, Selective plasma hydrolysis of glucocorticoid gamma-lactones and cyclic carbonates by the enzyme paraoxonase: an ideal plasma inactivation mechanism. J Med Chem 43, 19-21).

There is therefore a need for improved corticosteroid type compounds. In particular, it would be beneficial to provide useful prodrugs, which have limited activity (e.g. as anti-inflammatory or anticancer agents) until they are converted into the active form.

### BRIEF SUMMARY OF THE DISCLOSURE

The invention provides compounds comprising a monosaccharide or a disaccharide and an anti-inflammatory moiety or chemotherapeutic moiety, as well as a pharmaceutically acceptable salt, stereoisomer, acid, or solvate thereof. The compounds may be a substrate for glucosylceramidase beta 2 (GBA2), such that said compound is a prodrug that provides an active compound after cleavage of the monosaccharide or disaccharide by GBA2.

We have noted that such glycosylated compounds may be relatively inactive until their saccharide groups are removed, e.g. by GBA2. The enzyme GBA2 is preferentially expressed in inflamed tissues (and many cancer tissues also exhibit inflammation). This means that anti-inflammatory compounds and chemotherapeutic compounds may be converted into prodrugs by addition of a monosaccharide or a disaccharide moiety, which may be cleaved in or near a site of inflammation by GBA2, providing an active anti-inflammatory compound or chemotherapeutic compound in the inflamed tissue. This provides a novel approach to target therapeutic activity to the vicinity of the target tissue, thereby reducing off-target effects.

The invention provides in a first aspect a compound of formula I, or a pharmaceutically acceptable salt, stereoisomer, acid, or solvate thereof:

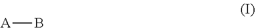

A is selected from a monosaccharide and a disaccharide. B is a therapeutic moiety, and may suitably be selected from an anti-inflammatory moiety or a chemotherapeutic moiety.

A second aspect of the invention provides a pharmaceutical composition comprising a compound of the first aspect. The composition may be formulated for non-oral administration. For example, the composition may be formulated for parenteral administration, e.g. the composition may be formulated for injection.

A third aspect provides a compound of the first aspect or a composition of the second aspect for use as a medicament. The medicament may be for use in the treatment of a condition associated with inflammation. The medicament may be for use in the treatment of a condition associated with inflammation by localised conversion of prodrug at the site of inflammation.

A fourth aspect provides a compound of the first aspect or a composition of the second aspect for use in the treatment of inflammation.

A fifth aspect provides a compound of the first aspect or a composition of the second aspect for use in the treatment of an inflammatory condition or other condition characterized by a hyperactivity of the immune system.

A sixth aspect provides a compound of the first aspect or a composition of the second aspect for use in the treatment of a condition characterized by hyperproliferation of cells belonging to the immune system.

A seventh aspect provides a compound of the first aspect or a composition of the second aspect for use in the treatment of cancer.

An eighth aspect provides use of a compound of the first aspect as an anti-inflammatory agent, comprising contacting the compound with cells that express a glucosylceramidase beta 2 (GBA2). The contacting may be performed in vitro. The contacting may be performed in vivo.

### BRIEF DESCRIPTION OF THE DRAWINGS

Embodiments of the invention are further described hereinafter with reference to the accompanying drawings, in which:

FIG. 1 shows the anti-inflammatory action of ginsenosides through activation of the GR. A. Schematic overview of the experimental approach used in the assays of the invention. Zebrafish larvae were subjected to tail wounding at 74 hpf. Chemical compound treatments started at 2 h before wounding and were continued for 4 h after wounding. At this time point, the number of neutrophils that had migrated to the wounded area (indicated by red box) was determined. B. Structures of the compounds used for treatment: Beclomethasone (Bec), Protopanaxadiol (PPD), F2 and Rb1. Glucose (Glc) groups conjugated to the steroid backbone are indicated in red. C. The number of migrated neutrophils upon compound treatment in wild type (gr^{+/+}) and Gr deficient (gr^{-/-}) individuals. D. Relative mRNA levels for il1b, il6 and mmp9, determined by qPCR, before and after wounding and upon compound treatment. E-H. The number of migrated neutrophils upon compound treatment in the absence and presence of the non-specific chemical Gba-inhibitor miglustat (E), in the absence and presence of the Gba1-inhibitor ME656 (F), the Gba2-inhibitor MZ31 (G), and in Gba2 deficient (gba2^{-/-}) individuals (H). Data shown are means±SEM of data pooled from three individual experiments in C and E-H), or the means±SEM of three individual experiments (in D). Statistical significance was determined by one- or two-way ANOVA and Tukey's post hoc test. Significant difference from the corresponding Veh group (grey bars, C, E, F, G, H) is indicated by *** (P<0.001), difference from the corresponding gr^{+/+} (C) or Same compound/Veh (E,G) group (same color bar, non-hatched) by ### (P<0.001), and difference from the Non-Wounding/Veh group (D) by +++ (P<0.001).

FIG. 2 shows the number of migrated neutrophils and macrophages in wild type zebrafish larvae upon treatment with Bec, PPD, F2 or Rb1. Data shown are means±SEM of data pooled from three individual experiments. Statistical significance was determined by one- or two-way ANOVA and Tukey's post hoc test. Significant difference from the Veh group is indicated by *** (P<0.001).

FIG. 3 shows a comparison of the side effects observed with beclomethasone relative to various ginsenosides. A. Whole body glucose levels, determined by ELISA, in 5 days post fertilization (dpf) zebrafish larvae, treated with beclomethasone or ginsenosides PPD, F2 or Rb1 from 2 hpf. B. Whole body glucose levels in 5 dpf larvae, upon wounding at 2 dpf, and compound treatment with or without MZ31. C. Whole body cortisol levels, determined by ELISA, in 5 days post fertilization (dpf) zebrafish larvae, treated with beclomethasone or ginsenosides PPD, F2 or Rb1 from 2 hpf. D. Whole body cortisol levels in 5 dpf larvae, upon wounding at 2 dpf, and compound treatment with or without MZ31. E. Body length of zebrafish larvae at 5 dpf upon compound treatment from 2 hpf. F. Length of regenerated tail fin of 5 dpf larvae, wounded at 2 dpf, upon compound treatment with or without MZ31. G. Relative GFP fluorescence level after 24 h compound treatment in 3 dpf larvae from the Tg(9x GCRE-HSV.UI23:EGFP) line, which is a reporter line for the transactivation activity of Gr. H. Representative brightfield microscopy images of tail fins of 5 dpf larvae from experimental groups presented in F. I. Representative fluorescence microscopy images of 3 dpf larvae from experimental groups presented in G. Data shown are means±SEM of three individual experiments. Statistical significance was determined by one- or two-way ANOVA and Tukey's post hoc test. Significant difference from the corresponding Veh group (A-F, H, grey bars) is indicated by * (P<0.05), ** (P<0.01), *** (P<0.001), difference from the Same compound/Veh group (E, G, same color bar, non-hatched) by #(P<0.05), ##(P<0.01), ### (P<0.001), and difference from the corresponding F2- and Rb1-treated groups (C, D, H) by +++ (P<0.001).

FIG. 4 shows further effects of treatment with beclomethasone relative to various ginsenosides on length of regenerated tail fins and mRNA levels in zebrafish larvae. A. Length of regenerated tail fins of 5 dpf larvae, wounded at 2 dpf, upon treatment with different concentrations of Bec. B. Relative mRNA levels for fkbp5, pck1 and nfkbiaa, determined by qPCR, before and after wounding and upon compound treatment. Data shown are means±SEM of three individual experiments. Statistical significance was determined by one- or two-way ANOVA and Tukey's post hoc test. Significant difference from the corresponding Veh group is indicated by *** (P<0.001), and difference from the PPD-, F2- and Rb1-treated groups is indicated by +++(P<0.001).

FIG. 5 shows the effects of wounding on mRNA levels for gba1 and gba2 in the tested zebrafish larvae. Shown are the relative mRNA levels for gba1 and gba2, determined by qPCR, in control larvae and at 4 h after wounding at 5 dpf. Data shown means±SEM three individual experiments. Statistical significance was determined by one- or two-way ANOVA and Tukey's post hoc test. Significant difference from the corresponding Control group is indicated by ** (P<0.01).

FIG. 6 shows the anti-inflammatory activity of glucoronidated dexamethasone (GDex) and side effects associated therewith compared with those of other compounds. A. The number of migrated neutrophils and macrophages in wild type zebrafish larvae upon treatment with Bec, dexamethasone (Dex), and glucuronidated dexamethasone (GDex) at 4 h after wounding. B. The number of migrated neutrophils upon compound treatment in Gr deficient (gr^{-/-}) individuals. C. Relative mRNA levels for il1b, il6 and mmp9 and mmp13, determined by qPCR, before and after wounding and upon compound treatment. D. Whole body glucose levels, determined by ELISA, in 5 days post fertilization (dpf) zebrafish larvae, treated with Bec, Dex, or G-Dex from 2 hpf. E. Whole body cortisol levels, determined by ELISA, in 5 days post fertilization (dpf) zebrafish larvae, treated with Bec, Dex, or GDex from 2 hpf. F. Length of regenerated tail fin of 5 dpf larvae, wounded at 2 dpf, upon compound treatment. G. Relative GFP fluorescence level after 24 h compound treatment in 3 dpf larvae from the Tg(9x GCRE-HSV.UI23:EGFP) line. H. Relative mRNA levels for fkbp5, pck1 and nfkbiaa, determined by qPCR, before and after wounding and upon compound treatment. Data shown are means±SEM of data pooled from three individual experiments (in A-B), or the means±SEM of three individual experiments (in C-H). Statistical significance was determined by one- or two-way ANOVA and Tukey's post hoc test. Significant difference from the corresponding Veh group is indicated by * (P<0.05), ** (P<0.01), *** (P<0.001) and difference from the Bec-(if present) and Dex-treated groups by +++ (P<0.001).

FIG. 7 shows the anti-inflammatory activity of glucosidated prednisolone (GPdn) and side effects associated therewith compared with those of other compounds. A. The number of migrated neutrophils upon treatment with Bec, Dex, GDex, prednisolone (Pdn), and glucosidated prednisolone (GPdn) in the absence and presence of the Gba2 inhibitor MZ31. B. Whole body glucose levels, determined by ELISA, in 5 days post fertilization (dpf) zebrafish larvae, treated with Bec, Dex, G-Dex, Pdn, or G-Pdn from 2 hpf. C. Whole body glucose levels in 5 dpf larvae, upon wounding at 2 dpf, and compound treatment with or without MZ31. D. Whole body cortisol levels, determined by ELISA, in 5 days post fertilization (dpf) zebrafish larvae, treated with Bec, Dex, G-Dex, Pdn, or G-Pdn from 2 hpf. E. Whole body cortisol levels in 5 dpf larvae, upon wounding at 2 dpf, and compound treatment with or without MZ31. Data shown are means±SEM of data pooled from three individual experiments (in A), or the average of three individual experiments (performed in triplicate) in B-E. Statistical significance was determined by one- or two-way ANOVA and Tukey's post hoc test. Significant difference from the corresponding Veh group (grey bars) is indicated by * (P<0.05), *** (P<0.001), difference from the corresponding group treated with a non-glycoidlated from of a compound (Dex or Pdn) by +++ (P<0.001, and difference from the Veh group for MZ31-treated groups (same color, non-hatched bars) by # (P<0.05), ### (P<0.001).

FIG. 8 shows the anti-inflammatory activity of gentibiosidated prednisolone (GbPdn) and side effects associated therewith compared with those of glucosidated prednisolone and prednisolone. A. Structures of the compounds used for treatment: prednisolone (Pdn) glucosidated prednisolone (GPdn) and gentiobiosidated prednisolone (GdPdn). The glucose (Glc) and gentiobiose (Gb) groups conjugated to the steroid backbone of Pdn are indicated in red. B. The number of migrated neutrophils upon treatment with Pdn, GPdn and gentiobiosidated Pdn (GbPdn), in the absence and presence of the Gba2 inhibitor MZ31. C. Relative mRNA levels for il1b, il6 and il8, determined by qPCR, before and after wounding and upon compound treatment. D. Whole body glucose levels, determined by ELISA, in 5 dpf control and wounded zebrafish larvae, treated with Pdn, GPdn or GbPdn from 2 hpf. E. Whole body cortisol levels, determined by ELISA, in 5 dpf control and wounded zebrafish larvae, treated with Pdn, GPdn or GbPdn from 2 hpf. Data shown are means±SEM of data pooled from three individual experiments (in B), or the means±SEM of three individual experiments (in C-E). Statistical significance was determined by one- or two-way ANOVA and Tukey's post hoc test. Significant difference from the corresponding Veh group (grey bars) is indicated by * (P<0.05), ** (P<0.01), *** (P<0.001), difference from the corresponding Pdn-treated group (pink bars) by +++ (P<0.001), and difference from the Veh group for MZ31-treated group (B) and from the Control group for Wounding (D, E) (same color, non-hatched bars) by # (P<0.05), ## (P<0.01), ### (P<0.001).

FIG. 9 shows further effects of treating zebrafish larvae with gentibiosidated prednisolone (GbPdn), glucosidsated prednisolone (GPdn) and prednisolone (Pdn). A. Length of regenerated tail fins of 5 dpf larvae, wounded at 2 dpf, upon treatment with different concentrations of Pdn. B. Body length of zebrafish larvae at 5 dpf upon compound treatment from 2 hpf. C. Length of regenerated tail fins of 5 dpf larvae, wounded at 2 dpf, upon compound treatment with or without MZ31. D. Relative GFP fluorescence level after 24 h compound treatment in 3 dpf larvae from the Tg(9x GCRE-HSV.UI23:EGFP) line. E. Representative fluorescence microscopy images of 3 dpf larvae from selected experimental groups presented in D. F. Relative mRNA levels for fkbp5, pck1 and nfkbiaa, determined by qPCR, before and after wounding and upon compound treatment. Data shown are means±SEM of three individual experiments. Statistical significance was determined by one- or two-way ANOVA and Tukey's post hoc test. Significant difference from the corresponding Veh group (grey bars) is indicated by *** (P<0.001), difference from the Pdn-treated group (pink bars) by +++ (P<0.001), and difference from the corresponding MZ31-treated group (C, same color, non-hatched bars) by ### (P<0.001).

FIG. 10 shows the relative binding affinities determined in vitro of using the PolarScreen Glucocorticoid Receptor (GR) Competitor Assay. Fluorescence polarization levels are plotted, reflecting binding of a fluorescent ligand that is competed off the receptor by increasing concentrations of the compounds Dex, Pdn, G-Pdn and Gb-Pdn. IC50 levels for each compound are indicated.

FIG. 11 provides a schematic overview of the procedure followed in the collagen antibody-induced arthritis (CAIA) mouse model experiment. Mice received a CAIA-cocktail on day 1, followed by LPS treatment on day 4. Prednisolone (Pdn) or gentibiosidated prednisolone (GbPdn) treatment was given daily from day 7 until 15.

FIG. 12 illustrates the therapeutic effects obtained for prednisolone (Pdn) and gentibiosidated prednisolone (GbPdn) in the CAIA model. A. Arthritis scores of the mice determined daily from day 5 until 15. B. Serum IL-6 levels determined by ELISA at day 10 and 15. Statistically significant difference from the Veh group is indicated by *** (P<0.001).

### DETAILED DESCRIPTION

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### Definitions

The following explanations of terms and methods are provided to better describe the present disclosure and to guide those of ordinary skill in the art in the practice of the present disclosure.

The invention concerns amongst other things the treatment of a disease. The term “treatment”, and the therapies encompassed by this invention, include the following and combinations thereof: (1) hindering, e.g. delaying initiation and/or progression of, an event, state, disorder or condition, for example arresting, reducing or delaying the development of the event, state, disorder or condition, or a relapse thereof in case of maintenance treatment or secondary prophylaxis, or of at least one clinical or subclinical symptom thereof; (2) preventing or delaying the appearance of clinical symptoms of an event, state, disorder or condition developing in an animal (e.g. human) that may be afflicted with or predisposed to the state, disorder or condition but does not yet experience or display clinical or subclinical symptoms of the state, disorder or condition; and/or (3) relieving and/or curing an event, state, disorder or condition (e.g., causing regression of the event, state, disorder or condition or at least one of its clinical or subclinical symptoms, curing a patient or putting a patient into remission). The benefit to a patient to be treated may be either statistically significant or at least perceptible to the patient or to the physician. It will be understood that a medicament will not necessarily produce a clinical effect in each patient to whom it is administered; thus, in any individual patient or even in a particular patient population, a treatment may fail or be successful only in part, and the meanings of the terms “treatment” and “prophylaxis” and of cognate terms are to be understood accordingly. The compositions and methods described herein are of use for therapy and/or prophylaxis of the mentioned conditions.

The term “prophylaxis” includes reference to treatment therapies for the purpose of preserving health or inhibiting or delaying the initiation and/or progression of an event, state, disorder or condition, for example for the purpose of reducing the chance of an event, state, disorder or condition occurring. The outcome of the prophylaxis may be, for example, preservation of health or delaying the initiation and/or progression of an event, state, disorder or condition. It will be recalled that, in any individual patient or even in a particular patient population, a treatment may fail, and this paragraph is to be understood accordingly.

The term “alkyl” as used herein includes reference to a straight or branched chain alkyl moiety having up to 20 (e.g. 1, 2, 3, 4, 5, 6, 7, 8, 9, 10, 11, 12, 13, 14, 15, 16, 17, 18, 19 or 20) carbon atoms. The term includes reference to, for example, methyl, ethyl, propyl (n-propyl or isopropyl), butyl (n-butyl, sec-butyl or tert-butyl), pentyl, hexyl and the like. In particular, alkyl may be a “C1-C8 alkyl”, i.e. an alkyl having 1, 2, 3, 4, 5, 6, 7 or 8 carbon atoms; “C1-C6 alkyl”, i.e. an alkyl having 1, 2, 3, 4, 5 or 6 carbon atoms; “C1-C4 alkyl”, i.e. an alkyl having 1, 2, 3 or 4 carbon atoms; or a “C1-C3 alkyl”, i.e. an alkyl having 1, 2 or 3 carbon atoms. The term “lower alkyl” includes reference to alkyl groups having 1, 2, 3 or 4 carbon atoms.

The term “alkenyl” as used herein includes reference to a straight or branched chain alkenyl moiety having up to 20 (e.g. 2, 3, 4, 5, 6, 7, 8, 9, 10, 11, 12, 13, 14, 15, 16, 17, 18, 19 or 20) carbon atoms. The term includes reference to, for example, ethenyl, propenyl, butenyl, pentenyl, hexenyl and the like. In particular, alkenyl may be a “C3-C8 alkenyl”, i.e. an alkenyl having 3, 4, 5, 6, 7 or 8 carbon atoms; “C3-C6 alkyl”, i.e. an alkenyl having 3, 4, 5 or 6 carbon atoms; “C3-C4 alkyl”, i.e. an alkenyl having 3 or 4 carbon atoms; The term “lower alkenyl” includes reference to alkyl groups having 2, 3 or 4 carbon atoms. The alkenyl may be monounsaturated (i.e. comprise a single carbon carbon double bond) or polyunsaturated (i.e. comprise a two or more carbon carbon double bonds, e.g. 2, 3 or 4 carbon carbon double bonds). For example, an alkenyl may be an alkadienyl, alkatrienyl, etc.

The term “cycloalkyl” as used herein includes reference to an alicyclic moiety having 3, 4, 5 or 6 carbon atoms. The group may be a bridged or polycyclic ring system. More often cycloalkyl groups are monocyclic. This term includes reference to groups such as cyclopropyl, cyclobutyl, cyclopentyl, cyclohexyl and the like.

The term “heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S, and wherein the nitrogen and sulfur atoms may optionally be oxidized and the nitrogen heteroatom may optionally be quaternized. The heteroatom(s) O, N, P, S and Si may be placed at any interior position of the heteroalkyl group or at the position at which the alkyl group is attached to the remainder of the molecule. Examples include, but are not limited to, —CH2-CH2-O—CH3, —CH2-CH2-NH—CH3, —CH2-CH2-N(CH3)-CH3, —CH2-S—CH2-CH3, —CH2-CH2, —S(O)—CH3, —CH2-CH2-S(O)2-CH3, —CH═CH—O—CH3, —Si(CH3)3, —CH2-CH═N—OCH3, —CH═CH—N(CH3)-CH3, O—CH3, —O—CH2-CH3, and —CN. Up to two heteroatoms may be consecutive, such as, for example, —CH2-NH—OCH3 and —CH2-O—Si(CH3)3. Similarly, the term “heteroalkylene” by itself or as part of another substituent means a divalent radical derived from heteroalkyl, as exemplified, but not limited by, —CH2-CH2-S—CH2-CH2- and —CH2-S—CH2-CH2-NH—CH2-. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)2R′— represents both —C(O)2R′— and —R′C(O)2-. As described above, heteroalkyl groups, as used herein, include those groups that are attached to the remainder of the molecule through a heteroatom, such as —C(O)R′, —C(O)NR′, —NR′R″, —OR′, —SR′, and/or —SO2R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The term “heterocycloalkyl” as used herein includes reference to a saturated heterocyclic moiety having 3, 4, 5, 6 or 7 ring carbon atoms and 1, 2, 3, 4 or 5 ring heteroatoms selected from nitrogen, oxygen, phosphorus and sulphur. For example, a heterocycloalkyl may comprise 3, 4, or 5 ring carbon atoms and 1 or 2 ring heteroatoms selected from nitrogen and oxygen. The group may be a polycyclic ring system but more often is monocyclic. This term includes reference to groups such as azetidinyl, pyrrolidinyl, tetrahydrofuranyl, piperidinyl, oxiranyl, pyrazolidinyl, imidazolyl, indolizidinyl, piperazinyl, thiazolidinyl, morpholinyl, thiomorpholinyl, quinolizidinyl and the like.

The terms “halo” or “halogen” as used herein includes reference to F, Cl, Br or I, for example F, Cl or Br. In a particular class of embodiments, halogen is F or Cl, of which F is more common.

The terms “halo” or “halogen,” by themselves or as part of another substituent, mean, unless otherwise stated, a fluorine, chlorine, bromine, or iodine atom; for example a halo may be a fluorine, chlorine or bromine. Additionally, terms such as “haloalkyl,” are meant to include monohaloalkyl and polyhaloalkyl. For example, the term “haloalkyl” refers to an alkyl group where one or more hydrogen atoms are substituted by a corresponding number of halogens. For example, the term “halo(C1-C4)alkyl” is mean to include, but not be limited to, trifluoromethyl, 2,2,2-trifluoroethyl, 4-chlorobutyl, 3-bromopropyl, and the like.

The term “alkoxy” as used herein include reference to —O-alkyl, wherein alkyl is straight or branched chain and comprises 1, 2, 3, 4, 5, 6, 7 or 8 carbon atoms. In one class of embodiments, alkoxy has 1, 2, 3 or 4 carbon atoms, e.g. 1, 2 or 3 carbon atoms. This term includes reference to, for example, methoxy, ethoxy, propoxy, isopropoxy, butoxy, tert-butoxy, pentoxy, hexoxy and the like. The term “lower alkoxy” includes reference to alkoxy groups having 1, 2, 3 or 4 carbon atoms.

The term “haloalkoxy” as used herein refers to an alkoxy group where one or more hydrogen atoms are substituted by a corresponding number of halogens.

Each of the above terms (e.g., “alkyl,” “cycloalkyl,” “heteroalkyl,” and “alkoxyl”), unless otherwise noted, are meant to include both substituted and unsubstituted forms of the indicated radical. Where a substituent is R-substituted (e.g. an Rx-substituted alkyl, where “x” is an integer), the substituent may be substituted with one or more R groups as allowed by chemical valency rules where each R group is optionally different (e.g. an Rx-substituted alkyl may include multiple Rx groups wherein each Rx group is optionally different). Certain examples of substituents for each type of radical are provided below.

The term “substituted” as used herein in reference to a moiety means that one or more, especially 1 to 5, more especially 1, 2 or 3, of the hydrogen atoms in said moiety are replaced independently of each other by the corresponding number of the described substituents. Unless otherwise specified, exemplary substituents include —OH, —CN, —NH₂, —NH(C1-C6 alkyl), —N(C1-C4 alkyl)2, ═O, -halo, —C1-C6 alkyl, —C2-C6 alkenyl, —C1-C6 haloalkyl, —C1-C6 haloalkoxy and —C2-C6 haloalkenyl, —C1-C6 alkylcarboxylic acid (e.g. —CH₃COOH or —COOH). Where the substituent is a —C1-C6 alkyl or —C1-C6 haloalkyl, the C1-C6 chain is optionally interrupted by an ether linkage (—O—) or an ester linkage (—C(O)O—). Exemplary substituents for a substituted alkyl may include —OH, —CN, —NH₂, ═O, -halo, —CO2H, —C1-C6 haloalkyl, —C1-C6 haloalkoxy and-C2-C6haloalkenyl, —C1-C6 alkylcarboxylic acid (e.g. —CH3COOH or —COOH). For example, exemplary substituents for an alkyl may include —OH, —CN, —NH₂, ═O, -halo.

It will, of course, be understood that substituents are only at positions where they are chemically possible, the person skilled in the art being able to decide (either experimentally or theoretically) without inappropriate effort whether a particular substitution is possible. For example, amino or hydroxy groups with free hydrogen may be unstable if bound to carbon atoms with unsaturated (e.g. olefinic) bonds. Additionally, it will of course be understood that the substituents described herein may themselves be substituted by any substituent, subject to the aforementioned restriction to appropriate substitutions as recognised by the skilled person.

Where steric issues determine placement of substituents on a group, the isomer having the lowest conformational energy may be preferred.

Where a compound, moiety, process or product is described as “optionally” having a feature, the disclosure includes such a compound, moiety, process or product having that feature and also such a compound, moiety, process or product not having that feature. Thus, when a moiety is described as “optionally substituted”, the disclosure comprises the unsubstituted moiety and the substituted moiety.

Where two or more moieties are described as being “independently” or “each independently” selected from a list of atoms or groups, this means that the moieties may be the same or different. The identity of each moiety is therefore independent of the identities of the one or more other moieties.

The term “molecular radical” by itself or as part of another substituent means a monovalent radical derived from the indicated molecular species. For example, a molecular radical of a given molecular species may correspond to the radical that remains after abstraction of a hydrogen atom or hydroxyl group from the given molecular species.

The term “pharmaceutically acceptable” as used herein includes reference to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio. This term includes acceptability for both human and veterinary purposes.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The term “acid” in relation to saccharide comprising compounds disclosed herein is meant to include forms of the compounds where the saccharide (and/or at least one monosaccharide residue thereof) comprises a carboxylic acid. Such a carboxylic acid may form part of an aldonic acid or a uronic acid. An aldonic acid is obtained when the terminal aldehyde group in an aldo sugar is oxidized, e.g. oxidation of D-glucose at C1 yields D-gluconic acid. A uronic acid is obtained when the hydroxyl group furthest from the carbonyl group of the sugar has been oxidized to a carboxylic acid, e.g. oxidation of D-glucose yields corresponding uronic acid D-glucoronate. Exemplary saccharide acids include gluconic acid, glucuronic acid, galactonic acid, galacturonic acid, idonic acid, iduronic acid, altronic acid, and altruronic acid; as well as D- and L-enantiomers thereof (such as D-gluconic acid, D-glucuronic acid, D-galactonic acid, D-galacturonic acid, L-idonic acid, and L-iduronic acid, L-altronic acid, and L-altruronic acid).

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centres) or double bonds; the racemates, diastereomers, tautomers, geometric isomers and individual isomers are encompassed within the scope of the present invention. The compounds of the present invention do not include those which are known in the art to be too unstable to synthesize and/or isolate.

The term “prodrug” as used herein represents compounds which are transformed in vivo to the parent compound or other active compound, for example, by hydrolysis in blood. An example of such a prodrug is a pharmaceutically acceptable ester of a carboxylic acid. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987; H Bundgaard, ed, Design of Prodrugs, Elsevier, 1985; and Judkins, et al. Synthetic Communications, 26(23), 4351-4367 (1996); and The organic chemistry of drug design and drug action by Richard B Silverman in particular pages 497 to 546; each of which is incorporated herein by reference. Compounds of the invention may represent prodrugs (e.g. comprising a latent anti-inflammatory agent or latent anti-cancer agent), where hydrolysis of a monosaccharide or disaccharide moiety, e.g. by the action of GBA2, provides an active anti-inflammatory agent or active anti-cancer agent.

The term “pharmaceutical composition” as used herein includes reference to a composition comprising at least one active compound (or prodrug) and optionally one or more additional pharmaceutically acceptable ingredients, for example a pharmaceutically acceptable carrier. Unless the context indicates otherwise, all references to a “composition” herein are references to a pharmaceutical formulation.

The term “product” or “product of the invention” as used herein includes reference to any product containing a compound of the present invention. In particular, the term product relates to compositions or formulations containing a compound of the present invention, such as a pharmaceutical composition, for example.

The term “therapeutically effective amount” as used herein refers to an amount of a drug, or pharmaceutical agent that, within the scope of sound pharmacological judgment, is calculated to (or will) provide a desired therapeutic response in a mammal (animal or human). The therapeutic response may for example serve to cure, delay the progression of or prevent a disease, disorder or condition.

The term “GBA2” as used herein refers to the enzyme glucosylceramidase beta 2, as specified by the HUGO Gene Nomenclature Committee. This enzyme is a microsomal beta-glucosidase that is known to catalyze the hydrolysis of bile acid 3-O-glucosides. We have determined that the enzyme GBA2 is selectively expressed at sites of inflammation, for example in induced sites of inflammation in zebra fish. Compounds provided herein which comprise a therapeutic moiety, such as an anti-inflammatory moiety or chemotherapeutic moiety, covalently linked to a monosaccharide or a disaccharide (such as compounds of formula I or formula II) may have the monosaccharide or disaccharide hydrolysed by GBA2 activity. GBA2 may therefore selectively release an active therapeutic moiety, such as an anti-inflammatory agent or active chemotherapeutic moiety at a site of inflammation. The compounds of the invention are thus suitable for use in the targeted delivery of active therapeutic moieties (including, but not limited to, active anti-inflammatory agents or active chemotherapeutic moieties) to sites of inflammation. It will be readily appreciated that this targeted delivery may be of benefit in respect of a wide range of therapeutic moieties to sites where inflammation is occurring. Inflammation is a component of many pathological processes and conditions, and this approach can be used to deliver therapeutic moieties suitable for treatment of such pathologies (not just for the alleviation of the inflammatory response) by in situ generation of active drug from a prodrug compound of the invention.

Although much is unknown about which saccharides mammalian GBA2 can cleave from lipophilic compounds such as cholesterol and steroid hormones, in addition to the results provided in the present disclosure, a few findings demonstrate that there is a certain level of specificity which can be exploited. GBA2 has been shown to cleave beta-D-glucosylated compounds (i.e. compounds with a D-glucose group attached by a beta-glycoside bond) (Marques, et al. (2016). “Glucosylated cholesterol in mammalian cells and tissues: formation and degradation by multiple cellular beta-glucosidases.” J Lipid Res 57(3): 451-463). GBA2 also cleaves beta-D-galactosylated compounds (Akiyama, et al. (2020). “Glucocerebrosidases catalyze a transgalactosylation reaction that yields a newly-identified brain sterol metabolite, galactosylated cholesterol.” J Biol Chem 295(16): 5257-5277). In contrast, this is not true for beta-xylosidated compounds (Boer, et al. (2021). “Human glucocerebrosidase mediates formation of xylosyl-cholesterol by beta-xylosidase and transxylosidase reactions.” J Lipid Res 62: 100018), GBA2 may therefore be considered to be specific for aldohexoses. Within the group of aldohexoses, additional specificity may be possible, as observed in studies in which derivatives of the iminosugar deoxynojirimycin were tested as inhibitors of GBA2 (Wennekes, et al. (2010). “Dual-action lipophilic iminosugar improves glycemic control in obese rodents by reduction of visceral glycosphingolipids and buffering of carbohydrate assimilation.” J Med Chem 53(2): 689-698; Ghisaidoobe, et al. (2014). “Identification and development of biphenyl substituted iminosugars as improved dual glucosylceramide synthase/neutral glucosylceramidase inhibitors.” J Med Chem 57(21): 9096-9104; Lahav, et al. (2017). “A Fluorescence Polarization Activity-Based Protein Profiling Assay in the Discovery of Potent, Selective Inhibitors for Human Nonlysosomal Glucosylceramidase.” J Am Chem Soc 139(40): 14192-14197). For example, based on these studies, L-idose was suggested to form substrates for potent GBA2 cleavage as well.

Previous studies considered GBA2 to be an exoglycosidase, i.e., an enzyme only able to cleave a monosaccharide from a lipophilic compound. However, we have demonstrated in the present application for the first time that GBA2 can actually remove a disaccharide from a steroid molecule, thereby suggesting it is actually an endoglycosidase, being able to cleave a disaccharide moiety from lipophilic compounds, such as corticosteroids.

The term “corticosteroid” as used herein refers to a class of steroid hormones, including natural corticosteroids that are produced by vertebrates and synthetic analogues of such compounds. Similarly to other steroid molecules, the carbon atoms of a corticosteroid may be numbered, based on the following hypothetical parent skeletal structure:

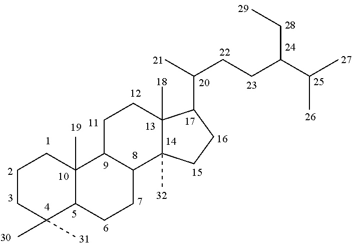

A preferred group of corticosteroids are glucocorticoids, which often have anti-inflammatory activity. Anti-inflammatory effects may be mediated by decreasing the levels of pro-inflammatory mediators (e.g. by transrepression of gene expression) and/or increasing the levels anti-inflammatory mediators (e.g. by transactivation of gene expression).

### Compounds

In one aspect, the invention provides compounds of formula I as previously described or a pharmaceutically acceptable salt, stereoisomer, solvate or prodrug thereof. These compounds may represent prodrugs, where the glycosylated compound has reduced biological activity compared to the corresponding aglycosylated (i.e. non-glycosylated) compound.

In the compound of formula I, A may be selected from glucose, galactose, mannose, fructose, and any acid form thereof, and any disaccharide formed from one or two saccharides selected from glucose, galactose, mannose, fructose, and any acid form thereof. The acid form of glucose, galactose, mannose, fructose may be an aldonic acid or a uronic acid; e.g. the acid form of glucose, galactose, mannose, fructose may be a uronic acid, such as glucuronic acid. An aldonic acid is obtained when the terminal aldehyde group in an aldo sugar is oxidized, e.g. oxidation of D-glucose at C1 yields D-gluconic acid. A uronic acid is obtained when the hydroxyl group furthest from the carbonyl group of the sugar has been oxidized to a carboxylic acid, e.g. oxidation of D-glucose yields corresponding uronic acid D-glucoronate.

In the compound of formula I, A may be an aldohexose. A may be selected from glucose, galactose, idose, altrose and any acid form thereof, and any disaccharide formed from one or two saccharides selected from glucose, galactose, idose, altrose, and any acid form thereof. The acid form of glucose, galactose, idose, altrose may be an aldonic acid or a uronic acid; e.g. the acid form of glucose, galactose, idose, altrose may be a uronic acid, such as glucuronic acid. An aldonic acid is obtained when the terminal aldehyde group in an aldo sugar is oxidized, e.g. oxidation of D-glucose at C1 yields D-gluconic acid. A uronic acid is obtained when the hydroxyl group furthest from the carbonyl group of the sugar has been oxidized to a carboxylic acid, e.g. oxidation of D-glucose yields corresponding uronic acid D-glucoronate. The position of the hydroxyl group at the C2 and/or C3 position may have an effect on GBA2 cleavage (van den Berg, et al. (2011). “Assessment of partially deoxygenated deoxynojirimycin derivatives as glucosylceramide synthase inhibitors.” ACS Med Chem Lett 2(7): 519-522). In view of this, A may be selected from D-glucose, D-galactose, L-idose, L-altrose and any acid form thereof, and any disaccharide formed from one or two saccharides selected from D-glucose, D-galactose, L-idose, L-altrose, and any acid form thereof. The acid form of D-glucose, D-galactose, L-idose, L-altrose may be an aldonic acid or a uronic acid; e.g. the acid form of D-glucose, D-galactose, L-idose, L-altrose, may be a uronic acid, such as glucuronic acid.

In the compound of formula I, A may be a disaccharide formed from one or two of each of glucose, galactose, mannose, fructose, and any acid form thereof (such as an aldonic acid or a uronic acid). A may be a disaccharide formed from one or two of each of glucose, galactose, mannose, fructose, and a uronic acid form thereof. A may be a disaccharide formed from one or two of each of glucose, galactose, mannose and fructose. For example, A may be gentiobiose. A may be a monosaccharide selected from glucose, galactose, mannose, fructose and any acid form thereof (such as an aldonic acid or a uronic acid). A may be a monosaccharide selected from glucose, galactose, mannose, fructose and a uronic acid form thereof. For example, A may be glucose or glucuronic acid. A may be a monosaccharide selected from glucose, galactose, mannose and fructose. For example, A may be glucose.

In the compound of formula I, A may be a disaccharide formed from two aldohexoses. For example, A may be a disaccharide formed from one or two of each of glucose, galactose, idose, altrose, and any acid form thereof (such as an aldonic acid or a uronic acid). A may be a disaccharide formed from one or two of each of glucose, galactose, idose, altrose, and a uronic acid form thereof. A may be a disaccharide formed from one or two of each of glucose, galactose, idose, and altrose. For example, A may be gentiobiose. The position of the hydroxyl group at the C2 and/or C3 position may have an effect on GBA2 cleavage (van den Berg, et al. (2011).supra). In view of this, A may be a disaccharide formed from one or two of each of D-glucose, D-galactose, L-idose, L-altrose, and any acid form thereof (such as an aldonic acid or a uronic acid). A may be a disaccharide formed from one or two of each of D-glucose, D-galactose, L-idose, L-altrose, and a uronic acid form thereof. A may be a disaccharide formed from one or two of each of D-glucose, D-galactose, L-idose, and L-altrose. For example, A may be gentiobiose.

In some embodiments disaccharides are preferred, as a disaccharide prodrug may have reduced activity compared a corresponding monosaccharide prodrug, while both compounds would provide the same active aglycosylated compounds. Thus the disaccharide and monosaccharide comprising compounds should provide similar levels of on-target activity (after the glycan has been removed), while the disaccharide may provide less side effects from off-target activity.

In the compound of formula I, B may be any therapeutic moiety that can be rendered less biologically active by addition of a monosaccharide or disaccharide as considered above. Exemplary categories of therapeutic moieties include an anti-inflammatory moiety, a chemotherapeutic moiety, an anti-infective moiety, an analgesic moiety, an anti-angiogenic moiety, and an antifibrotic moiety.

Suitably, B may be an anti-inflammatory moiety. The anti-inflammatory moiety may be or comprise a corticosteroid (e.g. a glucocorticoid), a non-steroidal glucocorticoid receptor agonist, a non-steroidal anti-inflammatory, a disease modifying antirheumatic drug, or rapamycin. The anti-inflammatory moiety may be or comprise a corticosteroid (e.g. a glucocorticoid). The anti-inflammatory moiety may be or comprise a non-steroidal glucocorticoid receptor agonist. The anti-inflammatory moiety may be or comprise a non-steroidal anti-inflammatory. The anti-inflammatory moiety may be or comprise a disease modifying antirheumatic drug. The anti-inflammatory moiety may be or comprise rapamycin.

The corticosteroid may be selected from a hydrocortisone type corticosteroid, a triamcinolone acetonide type corticosteroid, a methasone type corticosteroid, a betamethasone dipripionate type corticosteroid, and a methylprednisolone aceponate type corticosteroid. The corticosteroid may be a hydrocortisone type corticosteroid. The corticosteroid may be a triamcinolone acetonide type corticosteroid. The corticosteroid may be a methasone type corticosteroid. The corticosteroid may be a betamethasone dipripionate type corticosteroid. The corticosteroid may be a methylprednisolone aceponate type corticosteroid. These classes of corticosteroids are defined further below.

Based on cross-sensitivity in allergic contact dermatitis from corticosteroids, four structural classes of corticosteroid drugs have been defined (Coopman and Dooms-Goossens, Cross-reactions in topical corticosteroid contact dermatitis. Contact Dermatitis. 1988 August; 19(2):145-6; Coopman et al., Identification of cross-reaction patterns in allergic contact dermatitis from topical corticosteroids. Br J Dermatol. 1989 July; 121(1):27-34; Isaksson, Corticosteroids. Dermatol Ther. 2004; 17(4):314-20; Jacob and Steele, Corticosteroid classes: a quick reference guide including patch test substances and cross-reactivity. J Am Acad Dermatol. 2006 April; 54(4):723-7.). A description and generic structure is given for each class of corticosteroids in the following paragraphs. It should be noted that, in these paragraphs relating to classes of corticosteroids, a reference to “Cnn”, where “nn” is a number, is a reference to the indicated numbered carbon atom of the corticosteroid.

Class A: Hydrocorticone type corticosteroids are corticosteroids without substitution on the corticosteroid D-ring or C11 carbon chain, but including C11 and/or C21 acetate esters and the C21 thio-ester tixocortol pivalate.

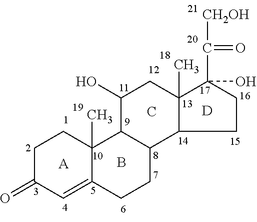

Class B: Triamcinolone acetonide type corticosteroids are corticosteroids with a C16, C17-cis, -diol, or -ketal chain structure.

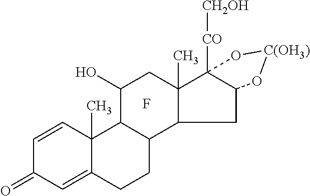

Class C: Betamethasone type corticosteroids are Corticosteroids with a C16 alkyl substitution.

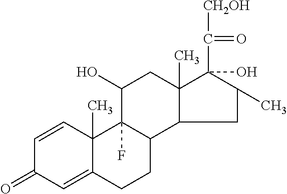

Class D: Hydrocortisone-17-butyrate type corticosteroids are corticosteroids with a long-chain ester at C17 and/or C21.

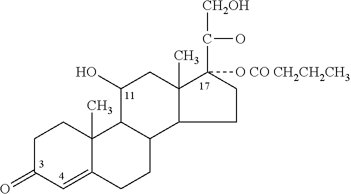

Class D can be further divided in two subclasses (Matura and Goossens, Reactions to corticosteroids: some new aspects regarding cross-sensitivity. Cutis. 2000 January; 65(1):43-5; Goossens et al., Contact allergy to corticosteroids. Allergy. 2000 August; 55(8):698-704), Classes D1 and D2. Class D1, Betamethasone dipropionate type corticosteroids, comprises corticosteroids with a methyl substitution on C16 and halogenation on B ring, and a long side chain ester on C17, and/or on C21. Class D2, Methylprednisolone aceponate type corticosteroids, comprises corticosteroids without a methyl substitution on C16, no halogenation, a long side chain ester on C17, and possibly a side chain on C21.

In the compound of formula I, B may be or comprise a molecular radical of alclometasone, amcinonide, beclomethasone, betamethasone, budesonide, chloroprednisone, ciclometasone, ciclesonide, clobetasol, clobetasone, clocortolone, cloprednol, cortifen, cortisol (hydrocortisone), cortisone, cortivazol, cortodoxone, deflazacort, desonide, desoximetasone, dexamethasone, dichlorisone, diflucortolone, diflorasone, difluprednate, formocortal, fluazacort, flucloronide, fludrocortisone, flucortin, flucortolone, flumetasone, flunisolide, fluocinolone, flucinolone acetonide, fluocinonide, fluocortin, fluorometholone, fluperolone, fluprednidene, fluprednisolone, flurandrenolide, fluticasone, halcinonide, halobetasol, halometasone, hydrocortamate, icometasone, isofluprednone, lotoprednol, mazipredone, medrysone, meprednisone, methylprednisolone, mometasone, nicocortonide, paramethasone, prednicarbate, prednisolone, prednimustine, prednisone, procinonide, tixocortol, triamcinolone or triamcinolone acetonide, or any esters thereof. The ester may be or comprise a group selected from an acetate, adamantoate, benzoate, benzofurancarboxylate, benzoyl-β-aminoisobutyrate, butyrate, butylate, caproate, carboxylate, chlorambucil, chlorphenacyl, cipecilate, cyclopentanepropionate, cyclopentylpropionate, cyclopropylcarboxylate, cypionate, dicibate, dicloacetate, diethylaminoacetate, enanthate, enbutate, etabonate (ethylcarbonate), fuorate, hemisuccinate, hexanoate, isobutyrate, isonicotinate, linoleate, metasulphobenzoate, metembonate, palmitate, phosphate, piperidinoacetate, pivalate, propionate, salicylate, stearoylglycolate, succinate, suleptanate, sulfate, tebutate (tert-butylacetate), tetrahydrophthalate, troxundate, valerate, or xanthogenic acid group.

In the compound of formula I, B may be a chemotherapeutic moiety. The chemotherapeutic moiety may be or comprise a molecular radical of alkylating agents (such as cisplatin), nitrosoureas (such as carmustine), antimetabolites (such as methotrexate and pentostatin), antitumor antibiotics (such as doxorubicin), topoisomerase inhibitors (such as irinotecan), and mitotic inhibitors (such as paclitaxel), or other chemotherapy drugs that do not fit well into any of these categories.

In the compound of formula I, B may be an anti-infective agent. In the compound of formula I, B may be an analgesic agent. In the compound of formula I, B may be an anti-angiogenic agent. In the compound of formula I, B may be an anti-fibrotic agent.

In an embodiment, the invention provides a compound of formula II, or a pharmaceutically acceptable salt, stereoisomer, acid, or solvate thereof:

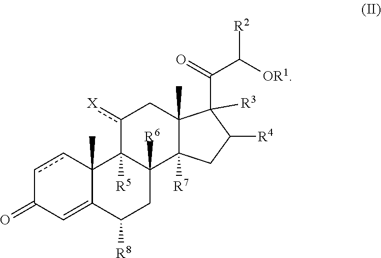

R¹ is selected from a monosaccharide or disaccharide. R² is selected from H, substituted or unsubstituted C₁-C₈ alkyl, substituted or unsubstituted C₁-C₈ alkoxy, substituted or unsubstituted C₃-C₈ alkenyl. R³ and R⁴ are each independently selected from H, OH, C₁-C₄ substituted or unsubstituted alkyl, OR⁹, OC(O)R⁹; or R³ and R⁴ together with the carbon atoms to which they are attached form a substituted or unsubstituted 5 or 6 membered heterocycloalkyl. R⁵, R⁶, R⁷ and R⁸ are each independently selected from H, OH, halo, substituted or unsubstituted —C₁-C₄ alkyl, or OR¹⁰. R⁹ is selected from H, or —C₁-C₈ substituted or unsubstituted alkyl. X is selected from ═O or —OH.

X may be ═O. X may be —OH.

The compound may be a compound of formula IIIa or IIIb, or a pharmaceutically acceptable salt, stereoisomer, acid, or solvate thereof:

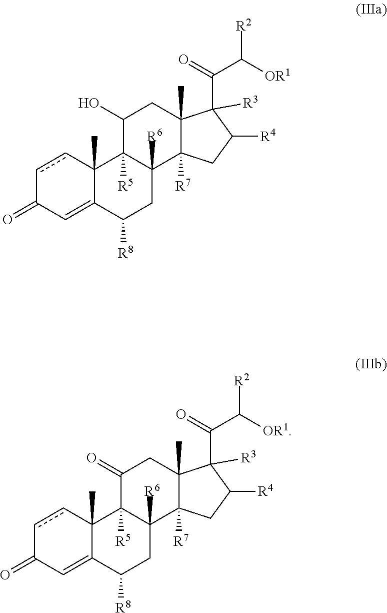

The compound may be a compound of formula IVa or IVb, or a pharmaceutically acceptable salt, stereoisomer, or solvate thereof:

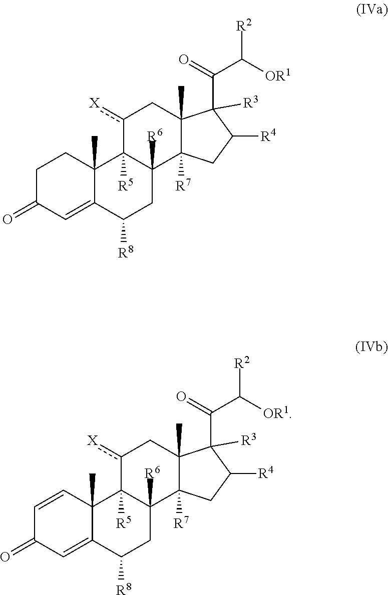

The compound may be a compound of formula Va, Vb, Vc or Vd, or a pharmaceutically acceptable salt, stereoisomer, acid, or solvate thereof:

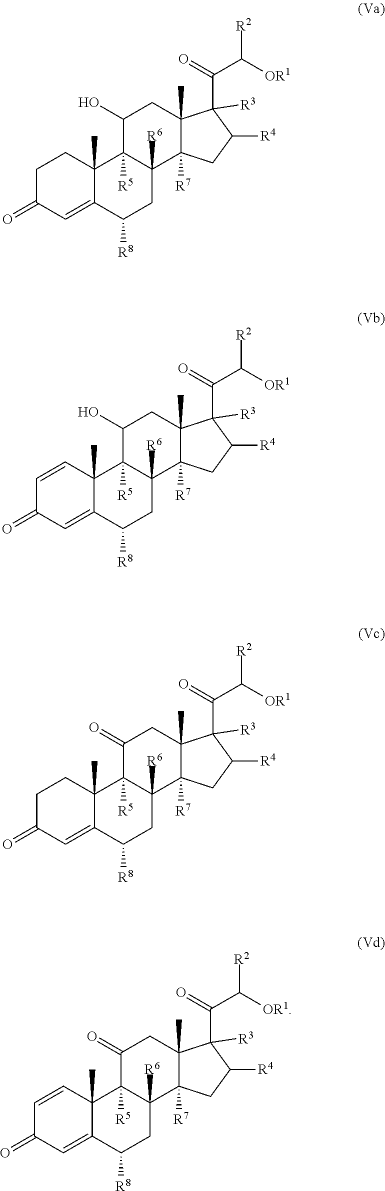

In each of formulae IIIa, IIIb, IVa, IVb, Va, Vb, Vc and Vd, the substituents are defined as follows. R¹ is selected from a monosaccharide or disaccharide. R² is selected from is aH, substituted or unsubstituted C₁-C₈ alkyl, substituted or unsubstituted C₁-C₈ alkoxy, substituted or unsubstituted C₃-C₈ alkenyl. R³ and R⁴ are each independently selected from H, OH, C₁-C₄ substituted or unsubstituted alkyl, OR⁹, OC(O)R⁹; or R³ and R⁴ together with the carbon atoms to which they are attached form a substituted or unsubstituted 5 or 6 membered heterocycloalkyl. R⁵, R⁶, R⁷ and R⁸ are each independently selected from H, OH, halo, substituted or unsubstituted —C₁-C₄ alkyl, or OR¹⁰. R⁹ is selected from H, or —C₁-C₈ substituted or unsubstituted alkyl.

In any of formulae II, IIIa, IIIb, IVa, IVb, Va, Vb, Vc and Vd, the substituents may be further defined as follows:
R¹ may be selected from glucose, galactose, mannose, fructose, and any acid form thereof, and any disaccharide formed from one or two saccharides selected from glucose, galactose, mannose, fructose, and any acid form thereof. The acid form of glucose, galactose, mannose, fructose may be an aldonic acid or a uronic acid; e.g. the acid form of glucose, galactose, mannose, fructose may be a uronic acid, such as glucuronic acid.

R¹ may be selected from an aldohexose, and any disaccharide formed from two aldohexoses (which may be the same or different). For example, R¹ may be selected from glucose, galactose, idose, altrose, and any acid form thereof, and any disaccharide formed from one or two saccharides selected from glucose, galactose, idose, altrose, and any acid form thereof. The acid form of glucose, galactose, idose, altrose, may be an aldonic acid or a uronic acid; e.g. the acid form of glucose, galactose, idose, altrose, may be a uronic acid, such as glucuronic acid. The position of the hydroxyl group at the C2 and/or C3 position may have an effect on GBA2 cleavage (van den Berg, et al. (2011), supra). In view of this, R¹ may be selected from D-glucose, D-galactose, L-idose, L-altrose, and any acid form thereof, and any disaccharide formed from one or two saccharides selected from D-glucose, D-galactose, L-idose, L-altrose, and any acid form thereof. The acid form of D-glucose, D-galactose, L-idose, L-altrose may be an aldonic acid or a uronic acid; e.g. the acid form of D-glucose, D-galactose, L-idose, L-altrose may be a uronic acid, such as glucuronic acid.

R¹ may be a disaccharide formed from one or two of each of glucose, galactose, mannose, fructose, and any acid form thereof (such as an aldonic acid or a uronic acid). R¹ may be a disaccharide formed from one or two of each of glucose, galactose, mannose, fructose, and a uronic acid form thereof. R¹ may be a disaccharide formed from one or two of each of glucose, galactose, mannose and fructose. For example, R¹ may be gentiobiose. R¹ may be a monosaccharide selected from glucose, galactose, mannose, fructose and any acid form thereof (such as an aldonic acid or a uronic acid). R¹ may be a monosaccharide selected from glucose, galactose, mannose, fructose and a uronic acid form thereof. For example, R¹ may be glucose or glucuronic acid. R¹ may be a monosaccharide selected from glucose, galactose, mannose and fructose. For example, R¹ may be glucose.

R¹ may be a disaccharide formed from two aldohexoses (which may be the same or different). R¹ may be a disaccharide formed from one or two of each of glucose, galactose, idose, altrose, and any acid form thereof (such as an aldonic acid or a uronic acid). R¹ may be a disaccharide formed from one or two of each of glucose, galactose, idose, altrose, and a uronic acid form thereof. R¹ may be a disaccharide formed from one or two of each of glucose, galactose, idose, and altrose. For example, R¹ may be gentiobiose. The position of the hydroxyl group at the C2 and/or C3 position may have an effect on GBA2 cleavage (van den Berg, et al. (2011), supra). In view of this, R¹ may be a disaccharide formed from one or two of each of D-glucose, D-galactose, L-idose, L-altrose, and any acid form thereof (such as an aldonic acid or a uronic acid). R¹ may be a disaccharide formed from one or two of each of D-glucose, D-galactose, L-idose, L-altrose, and a uronic acid form thereof. R¹ may be a disaccharide formed from one or two of each of D-glucose, D-galactose, L-idose, and L-altrose. For example, R¹ may be gentiobiose.

Compounds where R¹ is a disaccharide are sometimes preferred, as such compounds may represent disaccharide prodrugs that have reduced activity compared to the corresponding monosaccharide prodrug, while both compounds would provide the same active aglycosylated compounds. Thus the disaccharide and monosaccharide comprising compounds should provide similar levels of on-target activity (after the monosaccharide or disaccharide moiety has been removed), while the compounds where R¹ is a disaccharide may provide less side effects from off-target activity.

R² may be selected from H, and substituted or unsubstituted C₁-C₈ alkyl. R² may be H. R² may be substituted or unsubstituted C₁-C₈ alkyl. R² may be a substituted C₁-C₈ alkyl. R² may be an unsubstituted C₁-C₈ alkyl.

R³ may be selected from H, OH, and OC(O)R⁹. R³ may be selected from H and OH. R³ may be selected from H and OC(O)R⁹. R³ may be selected from OH, and OC(O)R⁹. R³ may be H. R³ may be OH. R³ may be OC(O)R⁹.

R⁹ may be a substituted or unsubstituted —C₁-C₈ alkyl. R⁹ may be a substituted —C₁-C₈ alkyl. R⁹ may be an unsubstituted —C₁-C₈ alkyl.

R⁴ may be selected from H, OH, or C₁-C₄ alkyl. R⁴ may be selected from H, OH, or methyl. R⁴ may be H. R⁴ may be OH. R⁴ may be C₁-C₄ alkyl (e.g. methyl).

R³ and R⁴ together with the carbon atoms to which they are attached may form a substituted or unsubstituted 5 or 6 membered heterocycloalkyl. For example, R³ and R⁴ together with the carbon atoms to which they are attached may form a substituted or unsubstituted 1,3 dioxolane. R³ and R⁴ together with the carbon atoms to which they are attached may form a substituted 1,3 dioxolane. The 1,3 dioxolane may be substituted at the 2 position with 1 or 2 groups selected from a —C₁-C₄ alkyl (such as methyl), a 5 or 6 membered cycloalkyl, or a 5 or 6 membered heterocyclic (such as furan). The 1,3 dioxolane may be substituted at the 2 position with 1 or 2 groups selected from a —C₁-C₄ alkyl, e.g. 1 or 2 methyl groups. The 1,3 dioxolane may be substituted at the 2 position with a 5 or 6 membered cycloalkyl. The 1,3 dioxolane may be substituted at the 2 position by a 5 or 6 membered heterocyclic, e.g. a furan.

In certain compounds, R² is H, R³ is OH, and R⁴ is H.

In certain compounds, R² is H, and R³ and R⁴ together with the carbon atoms to which they are attached form a substituted or unsubstituted 1,3 dioxolane.

In certain compounds, R² is H, and R⁴ is C₁-C₄ alkyl (such as methyl).

In certain compounds, R² is H, R³ is OC(O)R⁹, and R⁴ is H; optionally wherein R⁹ is substituted or unsubstituted —C₁-C₈ alkyl.

R⁵ may be selected from H and halo. R⁵ may be selected from H, F and Cl. R⁵ may be H. R⁵ may be F. R⁵ may be Cl.

R⁶ and R⁷ may each be H.

R⁸ may be selected from H and halo. R⁸ may be selected from H, F and Cl. R⁸ may be H. R⁸ may be F. R⁸ may be Cl.

R¹⁰ may be selected from H, or —C₁-C₈ (e.g. —C₁-C₄) substituted or unsubstituted alkyl. R¹⁰ may be H. R¹⁰ may be —C₁-C₆ (e.g. —C₁-C₄) substituted or unsubstituted alkyl.

The compound may be selected from:

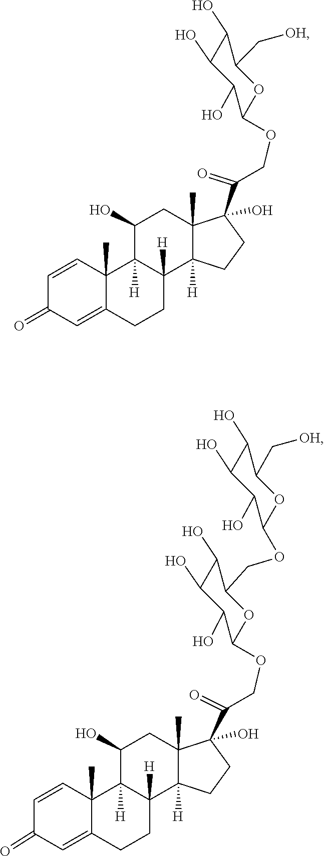

pharmaceutically acceptable salt, stereoisomer, acid, or solvate thereof.

The compound may be a substrate for glucosylceramidase beta 2 (GBA2).

A compound that is a substrate for GBA2 will have its saccharide groups hydrolyzed by the activity of the enzyme GBA2. GBA2 activity may be determined using two types of approach, a genetic and a pharmacological one, respectively, to 1) determine whether a specific effect of this compound is dependent on the presence and/or activity of this enzyme, and 2) determine in vitro whether a compound is converted by GBA2.

For 1) To determine whether a specific effect of a compound is dependent on the presence and/or activity of GBA2, this effect may be studied in a) genetically modified cultured cells or animal models (e.g. mouse or zebrafish (Yildiz et al., Mutation of beta-glucosidase 2 causes glycolipid storage disease and impaired male fertility. J Clin Invest. 2006 November; 116(11):2985-94; Lelieveld et al., Role of β-glucosidase 2 in aberrant glycosphingolipid metabolism: model of glucocerebrosidase deficiency in zebrafish. J Lipid Res. 2019 November; 60(11):1851-1867)) that are GBA2-deficient as a result of this modification, or b) in cultured cells or animal models in the presence of a chemical inhibitor of GBA2 (e.g. MZ31/L-ido-AMO-DNM (Wennekes et al., Dual-action lipophilic iminosugar improves glycemic control in obese rodents by reduction of visceral glycosphingolipids and buffering of carbohydrate assimilation. J Med Chem. 2010 Jan. 28; 53(2):689-98; Marques et al., Reducing GBA2 Activity Ameliorates Neuropathology in Niemann-Pick Type C Mice. PLoS One. 2015 Aug. 14; 10(8):e0135889)).

For 2) To determine in vitro whether a compound is converted by GBA2, a protocol is used that has previously been described by Lelieveld et al. (2019), supra; and Marques et al., Glucosylated cholesterol in mammalian cells and tissues: formation and degradation by multiple cellular beta-glucosidases. J Lipid Res. 2016 March; 57(3):451-63. Briefly, human cell cultures (e.g. HeLa or HEK293T) expressing GBA2 are grown. For the experiment, cells are harvested in phosphate-buffered saline (PBS), centrifuged and resuspended in in 25 mM potassium phosphate buffer supplemented with 0.1% (v/v) Triton-X100 and benzonase (25 mM Kpi pH 6.5, 0.1% Triton-X100 and 25 U/mL benzonase) and lysed using sonication. The cell homogenates are added to a mix of the compound in McIlvaine buffer of the appropriate pH and supplemented with additives (100 μL including 3.75 mM 4MU-B-Glc, 0.1% (w/v) BSA and 25 μM cholesterol with 1% ethanol in 150 mM McIlvaine pH 4 or pH 5.2 supplemented with 0.1% (v/v) Triton-X100 and/or 0.2% (w/v) Sodium Taurocholate). After 1 h incubation at 37° C. with shaking, 5 μL of the sample is transferred to 200 μL STOP buffer (1 M Glycine-NaOH, pH 10.3). Proteins are precipitated by addition of methanol and chloroform (2:1 (v/v)) and after centrifugation, lipids of the supernatant are extracted using a Bligh-Dyer extraction (MeOH:CHCl₃:H₂O, 1:1:0.9) and measured using liquid chromatography/mass spectrometry (LC/MS).

### Compositions and Administration

According to a further aspect of the invention there is provided a pharmaceutical composition comprising a compound of the invention. For example, the pharmaceutical composition may comprise a compound of any of formulae I, II, IIIa, IIIb, IVa, IVb, Va, Vb, Vc, or Vd. The composition may provide the compound in admixture with at least one pharmaceutically acceptable adjuvant, carrier, or diluent. The composition may be formulated for non-oral administration, in particular for non-oral systemic administration. The composition may be formulated for parenteral administration. The composition may be formulated for injection (e.g. for intravenous injection or intramuscular injection).

Compounds or compositions of the invention may be administered orally, topically, intravenously, subcutaneously, buccally, rectally, dermally, nasally, tracheally, bronchially, by any other parenteral route, as an oral or nasal spray or via inhalation. Preferably, the compounds or compositions are formulated for non-oral administration. The compounds may be administered in the form of pharmaceutical preparations comprising the compound either as a free compound or, for example, a pharmaceutically acceptable non-toxic organic or inorganic acid or base addition salt, in a pharmaceutically acceptable dosage form. Depending upon the disorder and patient to be treated and the route of administration, the compositions may be administered at varying doses.

Non-oral forms of administration may be preferred. The reason for this is that the glycan moiety (e.g. a monosaccharide or a disaccharide) of compounds of the invention may mask the activity of the remainder of the compounds of the disclosure. This glycan moiety is removed to provide an active aglycosylated moiety. In non-oral administration, this glycan removal may be performed at or proximate to a site of inflammation (e.g. via GBA2-catalysed hydrolysis). This provides an active compound at or proximate to the site of inflammation, with typically reduced activity remote from the site of inflammation, which can reduce side effects, especially when the compound is administered systemically. This advantage may be reduced if the compounds are administered orally, as the glycan moiety may be hydrolysed by the gut microbiota.

The pharmaceutical compounds of the invention may therefore be administered parenterally (“parenterally” as used herein, refers to modes of administration which include intravenous, intramuscular, intraperitoneal, intrasternal, subcutaneous and intraarticular injection and infusion) or orally to a host to obtain therapeutic effect (such as an anti-inflammatory or chemotherapeutic effect). For example, the pharmaceutical compounds of the invention may be administered by intravenous injection or infusion. In the case of larger animals, such as humans, the compounds may be administered alone or as compositions in combination with pharmaceutically acceptable diluents, excipients or carriers.

Actual dosage levels of active ingredients in the pharmaceutical formulations and pharmaceutical compositions of this invention may be varied so as to obtain an amount of the active compound(s) that is effective to achieve the desired therapeutic response for a particular patient, compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required to achieve the desired therapeutic effect and to gradually increase the dosage until the desired effect is achieved. Suitable doses are generally in the range of from 0.01-100 mg/kg/day, for example in the range of 0.1 to 50 mg/kg/day.

Pharmaceutical compositions of this invention for parenteral (e.g. intravenous) injection may comprise pharmaceutically acceptable sterile aqueous or non-aqueous solutions, dispersions, suspensions or emulsions as well as sterile powders for reconstitution into sterile injectable solutions or dispersions just prior to use. Examples of suitable aqueous and non-aqueous carriers, diluents, solvents or vehicles include water, ethanol, polyols (such as glycerol, propylene glycol, polyethylene glycol and the like), and suitable mixtures thereof, vegetable oils (such as olive oil) and injectable organic esters, such as ethyl oleate. Proper fluidity can be maintained, for example, by the use of coating materials, such as lecithin, by the maintenance of the required particle size in the case of dispersions and by the use of surfactants. Compositions for parenteral injection may represent preferred compositions of the invention.

These compositions may also contain adjuvants such as preservative, wetting agents, emulsifying agents and dispersing agents. Inhibition of the action of microorganisms may be ensured by the inclusion of various antibacterial and antifungal agents, for example, paraben, chlorobutanol or phenol sorbic acid. It may also be desirable to include isotonic agents, such as sugars or sodium chloride, for example. Prolonged absorption of the injectable pharmaceutical form may be brought about by the inclusion of agents (for example, aluminium monostearate and gelatine) which delay absorption.

Compositions for rectal or vaginal administration may be in the form of suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Dosage forms for topical administration of a compound of this invention include powders, sprays, creams, foams, gels, ointments and inhalants. The active compound is mixed under sterile conditions with a pharmaceutically acceptable carrier and any needed preservatives, buffers or propellants which may be required. Ophthalmic formulations, eye ointments, powders and solutions are also contemplated as being within the scope of this invention.

The compositions according to the present subject matter may also contain inactive components. Suitable inactive components are well known in the art and are described in standard textbooks, such as Goodman and Gillman's: The Pharmacological Bases of Therapeutics, 13^{th} Ed., Brunton et al., Eds. McGraw-Hill Education (2017), and Remington's Pharmaceutical Sciences, 17^{th} Ed., Mack Publishing Co., Easton, Pa. (1990), both of which are incorporated by reference herein in their entirety.

The compositions may be used in combination with an additional pharmaceutical dosage form to enhance their effectiveness in treating any of the disorders described herein. In this regard, the present formulations may be administered as part of a regimen additionally including any other pharmaceutical and/or pharmaceutical dosage form known in the art as effective for the treatment of any of these disorders.

### Uses

The compounds of the invention represent novel monosaccharide and disaccharide containing derivatives of therapeutic moieties, such as anti-inflammatories and chemotherapeutics. The compounds are therefore useful in the treatment of inflammation and /or cancer.

We have established that a therapeutic agent, such as an anti-inflammatory agent or anticancer agent can be converted into a prodrug by addition of a monosaccharide or disaccharide, to provide a compound comprising a glycan (monosaccharide or disaccharide) moiety and the therapeutic (e.g, anti-inflammatory moiety or chemotherapeutic) moiety.

GBA2, for example GBA2 present at or near a site of inflammation, is believed to hydrolyse the glycan moiety, providing an active aglyconic form of the therapeutic (e.g. anti-inflammatory or chemotherapeutic) moiety.

Inflammatory conditions are characterized by an excessive and/or uncontrolled and/or chronic inflammation (collectively referred to herein as “hyperactivity” of the immune system). Inflammation is a response of the body to harmful stimuli, such as pathogens, damaged cells, or irritants. It is a generic, non-specific response which involves the immune system. The five cardinal signs are heat, pain, redness, swelling, and loss of function. The function of inflammation is eradicating a pathogen or substance as well as damaged cells and tissues from the body, and to initiate tissue repair. However, excessive (often chronic) inflammation is associated with a number of clinical problems. It may become uncomfortable, it may prevent or delay normal progression of a healing response, it may protract injury, and it may result in various diseases. These include autoimmune diseases which arise from an abnormal immune response to a functioning body part, such as rheumatoid arthritis, allergies which arise from an excessive reaction to a foreign (often harmless) agent, such as hay fever, atopic dermatitis and allergic asthma, and inflammation related to infectious diseases such as COVID-19 and tuberculosis.

Conditions characterized by a hyperproliferation of cells belonging to the immune system include leukemia (acute lymphoblastic leukemia (ALL) and acute myeloid leukemia (AML)). ALL is a cancer of the lymphoid line of blood cells characterized by the development of large numbers of immature lymphocytes, whereas AML is a cancer of the myeloid line of blood cells, characterized by the rapid growth of abnormal cells that build up in the bone marrow and blood and interfere with normal blood cell production.

Furthermore, it should be noted that, as early as in the 19th century it was perceived that cancer is linked to inflammation (Balkwill et al., Smoldering and polarized inflammation in the initiation and promotion of malignant disease. Cancer Cell. 2005 March; 7(3):211-7; Mantovani et al., Cancer-related inflammation. Nature. 2008 Jul. 24; 454(7203):436-44; Multhoff et al., Chronic inflammation in cancer development. Front Immunol. 2012 Jan. 12; 2:98; Danforth, The Role of Chronic Inflammation in the Development of Breast Cancer. Cancers (Basel). 2021 Aug. 3; 13(15):3918). An inflammatory component is present in the microenvironment of most neoplastic tissues, including those not causally related to an obvious inflammatory process. Key features of cancer-related inflammation (CRI) include the infiltration of white blood cells, prominently tumor-associated macrophages (TAMs (Kimm et al., Tumor-Associated Macrophages-Implications for Molecular Oncology and Imaging. Biomedicines. 2021 Apr. 2; 9(4):374), which are likely to express GBA2. Without wishing to be bound by any theory, it is therefore submitted that glycosylated prodrugs of chemotherapeutic agents that are substrates of GBA2 (such as the compounds disclosed herein, e.g. of Formulae I and II), will induce a targeted activity on the tumor and its microenvironment where they are locally converted into the more active (non-glycosylated) form. This may result in a reduction of the side effects, when compared to direct administration of the corresponding non-glycosylated chemotherapeutic agent.

An aspect of the invention provides a compound of the invention or a pharmaceutical composition of the invention, for use as a medicament. The compound may, for example, be a compound of any of formulae I, II, IIIa, IIIb, IVa, IVb, Va, Vb, Vc, or Vd. The composition may comprise a compound any of formulae I, II, IIIa, IIIb, IVa, IVb, Va, Vb, Vc, or Vd. Such applications may make use of a compound of the invention incorporating any suitable therapeutic moiety that it is desired to provide to a site of inflammation. Suitably such applications may employ a compound of the invention incorporating an anti-inflammatory moiety or a chemotherapeutic moiety.

Inflammation and/or inflammatory conditions, which result in upregulation of GBA2, are associated with numerous pathologies. The compound of the present invention and disclosure (e.g. compounds of any of formulae I, II, IIIa, IIIb, IVa, IVb, Va, Vb, Vc, or Vd) or pharmaceutical compositions comprising such compounds may be used in the treatment of any such condition or pathology.

While the compounds of the invention are well suited to the delivery of anti-inflammatory agents, they may also be used for the targeted delivery of other therapeutic moieties to sites of inflammation. Since inflammation is found in many pathological processes and conditions, a wide range of therapeutic moieties may usefully be targeted in this manner.

Since inflammation is known to be associated with infection, compounds of the invention may comprise an anti-infective agent as a therapeutic moiety. Such compounds of the invention may be used as medicaments for the treatment of infection, and/or for the treatment of inflammation related complications of infection.

Since pain is known to be associated with many processes involving inflammation, compounds of the invention may comprise an analgesic agent as a therapeutic moiety. Such compounds of the invention may be used as medicaments for the alleviation of pain, particularly pain associated with inflammation.

Inflammation is also known to be associated with many processes in which excessive or pathological neovascularization occurs. Accordingly, compounds of the invention may comprise an anti-angiogenic agent as a therapeutic moiety. Such compounds of the invention may be used as medicaments for the treatment of conditions associated with excessive blood vessel formation. Merely by way of example, such conditions may include cancer, psoriasis, vascular retinopathies (such as proliferative diabetic retinopathy or wet macular degeneration), and wound healing.

The role of inflammation in wound healing lends itself to medical uses of compounds of the invention that comprise an anti-fibrotic agent as a therapeutic moiety. Such compounds of the invention may be used as medicaments for the inhibition of scarring.

Thus, suitable therapeutic moieties that may be incorporated in compounds of the invention are not limited to anti-inflammatory moieties or chemotherapeutic moieties, but may also include, merely by way of example, anti-fibrotic moieties, analgesic moieties, anti-angiogenic moieties, or anti-infective moieties.

The use in any of the applications considered above may comprise non-oral administration of the compound or the composition to a patient. The non-oral administration may comprise parenteral administration. For example, the non-oral administration may comprise injection.

An aspect of the invention provides a compound of the invention or a pharmaceutical composition of the invention, for use in the treatment of inflammation. This aspect also provides a compound of the invention, or a pharmaceutical composition of the invention, for use to alleviate inflammation. The compound may, for example, be a compound of any of formulae I, II, IIIa, IIIb, IVa, IVb, Va, Vb, Vc, or Vd. The composition may comprise a compound any of formulae I, II, IIIa, IIIb, IVa, IVb, Va, Vb, Vc, or Vd. Such applications may employ a compound of the invention incorporating an anti-inflammatory moiety.

The use may comprise non-oral administration of the compound or the composition to a patient. The non-oral administration may comprise parenteral administration. For example, the non-oral administration may comprise injection.

Another aspect of the invention provides a method for the treatment of inflammation, comprising administration (e.g. non-oral administration) of an effective amount of a compound of the invention or a pharmaceutical composition of the invention to a patient. The compound may, for example, be a compound of any of formulae I, II, IIIa, IIIb, IVa, IVb, Va, Vb, Vc, or Vd. The composition may comprise a compound any of formulae I, II, IIIa, IIIb, IVa, IVb, Va, Vb, Vc, or Vd. Such applications may employ a compound of the invention incorporating an anti-inflammatory moiety.

A related aspect provides use of a compound of the invention for the manufacture of a medicament for the treatment of inflammation.

An aspect of the invention provides a compound of the invention or a pharmaceutical composition of the invention, for use in the treatment of an inflammatory condition or other condition characterized by a hyperactivity of the immune system. The compound may, for example, be a compound of any of formulae I, II, IIIa, IIIb, IVa, IVb, Va, Vb, Vc, or Vd. The composition may comprise a compound any of formulae I, II, IIIa, IIIb, IVa, IVb, Va, Vb, Vc, or Vd. Such applications may employ a compound of the invention incorporating an anti-inflammatory moiety.

The inflammatory or other condition may be selected from arthrosis, osteoarthritis, gout, rheumatoid arthritis, other auto-immune disorders (such as systemic lupus erythematosus (SLE), inflammatory bowel disease (IBD, including Crohn's disease, ulcerative colitis), multiple sclerosis, chronic inflammatory demyelinating polyneuropathy (CIPD)), infectious disease (such as COVID-19 and tuberculosis), asthma, chronic obstructive pulmonary disease (COPD), allergic and non-allergic rhinitis and sinusitis, tendinitis, nasal polyps, skin conditions (such as rash, dermatitis, itching, eczema, dermatomycosis, lichen (sclerosus or ruber), and psoriasis), ocular disease (such as uveitis, conjunctivitis, macular edema), otitis, thyroiditis, sarcoidosis, myositis, vasculitis, haemorrhoids, organ rejection in transplant recipients and graft versus host disease.

The inflammatory or other condition may be selected from rheumatoid arthritis, other auto-immune disorders (such as systemic lupus erythematosus (SLE), inflammatory bowel disease (IBD)), thyroiditis, asthma, chronic obstructive pulmonary disease (COPD), allergic and non-allergic rhinitis, respiratory infection (e.g. COVID-19), nasal polyps, psoriasis, eczema, dermatitis, ocular disease, and graft versus host disease. The inflammatory or other condition may be selected from rheumatoid arthritis, other auto-immune disorders (such as systemic lupus erythematosus (SLE), inflammatory bowel disease (IBD)), and thyroiditis.

The use may comprise non-oral administration of the compound or the composition to a patient. The non-oral administration may comprise parenteral administration. For example, the non-oral administration may comprise injection.

Another aspect of the invention provides a method for the treatment of an inflammatory condition or other condition characterized by a hyperactivity of the immune system, comprising administration (e.g. non-oral administration) of an effective amount of a compound of the invention or a pharmaceutical composition of the invention to a patient. The compound may, for example, be a compound of any of formulae I, II, IIIa, IIIb, IVa, IVb, Va, Vb, Vc, or Vd. The composition may comprise a compound any of formulae I, II, IIIa, IIIb, IVa, IVb, Va, Vb, Vc, or Vd.

The inflammatory or other condition may be selected from arthrosis, osteoarthritis, gout, rheumatoid arthritis, other auto-immune disorders (such as systemic lupus erythematosus (SLE), inflammatory bowel disease (IBD, including Crohn's disease, ulcerative colitis), multiple sclerosis, chronic inflammatory demyelinating polyneuropathy (CIPD)), infectious disease (such as COVID-19 and tuberculosis), asthma, chronic obstructive pulmonary disease (COPD), allergic and non-allergic rhinitis and sinusitis, tendinitis, nasal polyps, skin conditions (such as rash, dermatitis, itching, eczema, dermatomycosis, lichen (sclerosus or ruber), and psoriasis), ocular disease (such as uveitis, conjunctivitis, macular edema), otitis, thyroiditis, sarcoidosis, myositis, vasculitis, haemorrhoids, organ rejection in transplant recipients and graft versus host disease.

The inflammatory or other condition may be selected from rheumatoid arthritis, other auto-immune disorders (such as systemic lupus erythematosus (SLE), inflammatory bowel disease (IBD)), thyroiditis, asthma, chronic obstructive pulmonary disease (COPD), allergic and non-allergic rhinitis, respiratory infection (e.g. COVID-19), nasal polyps, psoriasis, eczema, dermatitis, ocular disease, and graft versus host disease. The inflammatory or other condition may be selected from rheumatoid arthritis, other auto-immune disorders (such as systemic lupus erythematosus (SLE), inflammatory bowel disease (IBD)), and thyroiditis.

A related aspect provides use of a compound of the invention for the manufacture of a medicament for the treatment of an inflammatory condition or other condition characterized by a hyperactivity of the immune system. The inflammatory condition or other condition characterized by a hyperactivity of the immune system may be as further defined above.

An aspect of the invention provides a compound of the invention or a pharmaceutical composition of the invention, for use in the treatment of a condition characterized by hyperproliferation of cells belonging to the immune system. The compound may, for example, be a compound of any of formulae I, II, IIIa, IIIb, IVa, IVb, Va, Vb, Vc, or Vd. The composition may comprise a compound any of formulae I, II, IIIa, IIIb, IVa, IVb, Va, Vb, Vc, or Vd. Such applications may employ a compound of the invention incorporating a chemotherapeutic moiety.

The condition may be selected from acute lymphoblastic leukemia (ALL), and acute myeloid leukemia (AML).

The use may comprise non-oral administration of the compound or the composition to a patient. The non-oral administration may comprise parenteral administration. For example, the non-oral administration may comprise injection.

Another aspect of the invention provides a method for the treatment of a condition characterized by hyperproliferation of cells belonging to the immune system, comprising administration (e.g. non-oral administration) of an effective amount of a compound of the invention or a pharmaceutical composition of the invention to a patient. The compound may, for example, be a compound of any of formulae I, II, IIIa, IIIb, IVa, IVb, Va, Vb, Vc, or Vd. The composition may comprise a compound any of formulae I, II, IIIa, IIIb, IVa, IVb, Va, Vb, Vc, or Vd. Such applications may employ a compound of the invention incorporating a chemotherapeutic moiety.

The condition may be selected from acute lymphoblastic leukaemia (ALL), and acute myeloid leukaemia (AML).

A related aspect provides use of a compound of the invention for the manufacture of a medicament for the treatment of a condition characterized by hyperproliferation of cells belonging to the immune system. The condition characterized by hyperproliferation of cells belonging to the immune system may be as further defined above.

An aspect of the invention provides a compound of the invention or a pharmaceutical composition of the invention, for use in the treatment of cancer. The cancer may be selected from acute lymphoblastic leukaemia (ALL), and acute myeloid leukaemia (AML). The compound may, for example, be a compound of any of formulae I, II, IIIa, IIIb, IVa, IVb, Va, Vb, Vc, or Vd. The composition may comprise a compound any of formulae I, II, IIIa, IIIb, IVa, IVb, Va, Vb, Vc, or Vd. Such applications may employ a compound of the invention incorporating a chemotherapeutic moiety.

The use may comprise non-oral administration of the compound or the composition to a patient. The non-oral administration may comprise parenteral administration. For example, the non-oral administration comprises injection.

Another aspect of the invention provides a method for the treatment of cancer, comprising administration (e.g. non-oral administration) of an effective amount of a compound of the invention or a pharmaceutical composition of the invention to a patient. The cancer may be selected from acute lymphoblastic leukaemia (ALL), and acute myeloid leukaemia (AML). The compound may, for example, be a compound of any of formulae I, II, IIIa, IIIb, IVa, IVb, Va, Vb, Vc, or Vd. The composition may comprise a compound any of formulae I, II, IIIa, IIIb, IVa, IVb, Va, Vb, Vc, or Vd. Such applications may employ a compound of the invention incorporating a chemotherapeutic moiety.

A related aspect provides use of a compound of the invention for the manufacture of a medicament for the treatment of cancer. The cancer may be selected from acute lymphoblastic leukaemia (ALL), and acute myeloid leukaemia (AML). The compound may, for example, be a compound of any of formulae I, II, IIIa, IIIb, IVa, IVb, Va, Vb, Vc, or Vd. The composition may comprise a compound any of formulae I, II, IIIa, IIIb, IVa, IVb, Va, Vb, Vc, or Vd. Such applications may employ a compound of the invention incorporating a chemotherapeutic moiety.

An aspect of the invention provides use of a compound of the invention as an anti-inflammatory agent. The use comprises comprising contacting the compound with cells that express a glucosylceramidase beta 2 (GBA2). Such applications may employ a compound of the invention incorporating an anti-inflammatory moiety

The use may be an in vitro use. The cells may be in a tissue or organ. The cells may be in a cell culture. The compound may be a compound of any of formulae I, II, IIIa, IIIb, IVa, IVb, Va, Vb, Vc, or Vd. The composition may comprise a compound any of formulae I, II, IIIa, IIIb, IVa, IVb, Va, Vb, Vc, or Vd.

### Assays

Compounds of the invention can be assessed for biological activity using any suitable assay that would be known to the person skilled in the art. Exemplary assays that are useful for the assessment of compounds of the invention are provided in the following paragraphs.

### Materials and Methods

Zebrafish (Danio rerio) were maintained and handled according to the guidelines from the Zebrafish Model Organism Database (https://zfin.org/) and in compliance with the directives of the local animal welfare committee of Leiden University. To maintain circadian rhythmicity, adult fish were exposed to a 14 h light and 10 h dark cycle. Fertilization was performed by natural spawning at the beginning of the light period. Fertilized eggs were collected and raised at 28° C. in egg water (60 μg/ml Instant Ocean sea salts and 0.0025% methylene blue). The following zebrafish lines were used in this study: AB/TL wild type, the transgenic lines Tg(mpx:GFPⁱ¹¹⁴/mpeg1:mCherry^{umsF001}) (Bernut et al., (2014). Mycobacterium abscessus cording prevents phagocytosis and promotes abscess formation. Proc Natl Acad Sci USA 111, E943-952; Renshaw et al., (2006). A transgenic zebrafish model of neutrophilic inflammation. Blood 108, 3976-3978), and Tg(9xGCRE-HSV.UI123:EGFP)^{ia20} (Benato et al., (2014). A living biosensor model to dynamically trace glucocorticoid transcriptional activity during development and adult life in zebrafish. Mol Cell Endocrinol 392, 60-72), and the mutant lines gba2^{-/-} (Lelieveld et al., (2019). Role of beta-glucosidase 2 in aberrant glycosphingolipid metabolism: model of glucocerebrosidase deficiency in zebrafish. J Lipid Res 60, 1851-1867) and gr^{s357} (Ziv et al., (2013). An affective disorder in zebrafish with mutation of the glucocorticoid receptor. Mol Psychiatry 18, 681-691).

The compounds beclomethasone, prednisolone, dexamethasone, dexamethasone, PPD, Rb1, F2 and miglustat were purchased from Sigma-Aldrich (Burlington, MA, USA), and dexamethasone-glucuronide (referred to as glucoronidated dexamethasone, GDex) from Santa Cruz (Dallas, TX, USA), ME656 and MZ31 were kindly provided by Dr. Hans Aerts (Leiden University, The Netherlands). The compounds 21-O-Glc-prednisolone (glucosidated prednisolone, GPdn) and 21-O-Gb-prednisolone (gentiobiosidated prednisolone, GbPdn) were synthesized as described herein in the examples.

### Tail Fin Amputation Assay

For the tail fin amputation experiments, 3 days post fertilization (dpf) larvae from the Tg(mpx:GFPⁱ¹¹⁴/mpeg1:mCherry^{umsF001}) line were anesthetized in 0.02% buffered aminobenzoic acid ethyl ester (tricaine) in egg water and placed on a 2% agarose-coated petri dish. The tails were partially amputated with a 1 mm sapphire blade (World Precision Instruments) under a Leica M165C stereomicroscope (Leica Microsystems, Wetzlar, Germany). Chemical treatment (or 0.1% DMSO as vehicle treatment) was started at 2 h before the amputation (pretreatment) and was continued for 4 h after the tail fin amputation (20 larvae per group, unless indicated otherwise), at which time point the larvae were fixed in 4% paraformaldehyde (PFA) overnight at 4° C. and stored at 4° C. until further examination. In some experiments, a 24 h treatment with miglustat, ME656 or MZ31 was started at 2 dpf, and these treatments were continued during the pretreatment and treatment with the other chemicals. For imaging of larvae from the Tg(mpx:GFPⁱ¹¹⁴/mpeg1:mCherry^{umsF001}) line, a LeicaMZ16FA fluorescence stereomicroscope with LAS 3.7 software was used. Neutrophils were detected by GFP fluorescence and macrophages by mCherry fluorescence. The neutrophil numbers were determined by blinded manual counting in a pre-determined area. When larvae from gba2^{-/-} or gr^{s357}mutant lines were used, neutrophil labeling was performed with the TSA fluorescein detection kit (PerkinElmer) for specific staining of Myeloperoxidase (Mpx)-positive cells, following the manufacturer's instructions.

### Measurement of Larval Body Length and Regeneration of the Tail Fin

When the regeneration of the tail fins after wounding or the larval body length was used as a readout, chemical treatments of the embryos were started at 2 hours post fertilization (hpf) and continued until 5 dpf. During this period, solutions were refreshed daily. At 5 dpf, larvae were imaged using a LeicaMZ16FA fluorescence stereomicroscope supported by LAS 3.7 software, and the length of the larvae was measured using ImageJ software (15 larvae per treatment group). For the regeneration experiments, tail fins were amputated at 2 dpf and larvae were imaged at 5 dpf (15 larvae per treatment group). The newly grown tissue can be distinguished from the old tissue, enabling precise measurement of the size of the newly grown tissue using ImageJ software.

### Whole-Body Glucose Measurements Using a Colorimetric Assay

Zebrafish embryos at 2 hpf received a chemical treatment, as indicated, with daily refreshment of the solutions, until 5 dpf. At 5 dpf, the larvae were collected in an Eppendorf tube and 100 μL of ice-cold glucose buffer was added to each sample. The larvae were homogenized using a BulletBlender® for 3 min at 8,000 rpm. The homogenates were then centrifuged at 4° C. for 8 min at 11000 rpm and the supernatant was stored at −20° C. Whole-body glucose concentrations were determined using a Glucose Colorimetric Assay kit (Cayman Chemical, USA), according to the manufacturer's instructions. In each experiment, three biological replicates were used for each treatment group, and the colorimetric assay was performed using technical duplicates.

### Whole-Body Cortisol Measurements Using ELISA

Zebrafish embryos at 2 hpf received a chemical treatment, as indicated, with daily refreshment of the solutions, until 96 hpf. Then, the treatments were stopped and embryos were incubated in egg water until 5 dpf to avoid cross-reactivity with the compounds of the cortisol antibody used in the ELISA. Then, the treatments were stopped and replaced with egg water. At 5 dpf, the larvae were collected in an Eppendorf tube and 100 μL of ice-cold phosphate-buffered saline (PBS) was added. The larvae were homogenized using a BulletBlender® for 3 min at 8,000 rpm. Ethyl acetate was added to the homogenate and the supernatant was gathered and vaporized. A volume of 150 μl of 0.2% Bovine serum albumin (Sigma Aldrich) dissolved in phosphate-buffered saline (PBS) was added to the samples and frozen. Whole-body cortisol of the zebrafish larvae was determined by ELISA (Demeditec Diagnostics GmbH, Kiel-Wellsee, Germany), following the manufacturer's instructions. In each experiment, three biological replicates were used for each treatment group, and the colorimetric assay was performed using technical duplicates.

### Transactivation Activity of Gr in Zebrafish Reporter Line

The Tg(9xGCRE-HSV.UI23:EGFP)^{ia20} zebrafish reporter line expresses enhanced green fluorescent protein (EGFP) under the control of a promoter containing an array of nine GREs. To study the effect of chemical treatments on the transactivation activity of the Gr, 15 zebrafish embryos per group (2 dpf) were treated with the indicated compounds for 24 hours. The transactivation activity of Gr induced by the treatments was assessed using a LeicaMZ16FA fluorescence stereomicroscope supported by LAS 3.7 software. The integrated intensity of the EGFP signal in the larvae was determined using ImageJ software.

### Overexpression of Gba2 in Zebrafish

For overexpression of gba2 in zebrafish, a plasmid containing the cDNA encoding the zebrafish gba2 gene fused to a CMV promoter (pDEST-zeo-zGBA2 (Lelieveld et al., 2020) was used. One cell stage zebrafish embryos were injected with this plasmid, diluted in nuclease free water (1 nl/egg with final concentration of 80 pg/egg), using the Automated Microinjection System Version 3 AMS-03 (Life Science Methods BV). After the injection, chemical treatment was performed as indicated.

Quantitative PCR (qPCR) Analysis

For experiments in zebrafish, gene expression was measured using 3 dpf embryos. Groups of 15 larvae were collected in TRIzol reagent (Invitrogen) and mRNA was isolated using the miRNeasy mini kit (Qiagen), according to the manufacturer's instructions. For experiments in HeLa cells, samples were collected from a single well from 6 well plates (80% confluent) in TRIzol reagent and mRNA was isolated using the RNeasy mini kit. Zebrafish and cells samples were treated with DNAse utilizing the DNA-Free™ kit (Ambion). The cDNA synthesis was performed using iScript cDNA synthesis kit (Bio-Rad) using 1 μg of RNA per sample. For qPCR, 10 μM of forward and 10 μM of reverse primer, 12.5 μL of iQ SYBR Green Supermix (Bio-Rad), and 2 μL of cDNA were added to the qPCR reaction mixture. Each mixture had a total volume of 25 μL, which was split as duplicates with a volume of 12.5 μL. The qPCR reactions were performed on a MyiQ-single-color real-time PCR detection system (Bio-Rad) with initial denaturation for 3 min at 95° C. and 40 cycles of 15 s at 95.5° C., 15 s at 60° C., and 30 s at 72° C. Cycle threshold values (Ct values, i.e. the cycle numbers at which a threshold value of the fluorescence intensity was reached) were determined for each sample. The gene expression level for each sample was normalized using the expression of ppial (peptidylprolyl isomerase Ab (cyclophilin A)) for zebrafish samples, and 18S rRNA for human cells. The fold change per sample (compared to the respective control group) was calculated using the ΔΔCt method. In each experiment, biological triplicates three biological replicates were used for each treatment group, and reactions were performed in duplicates.

Cell Culture, Transfection and shRNA Gene Knockdown

HeLa cells (purchased from ATCC) were cultured in Dulbecco's Modified Eagle's (DMEM) High Glucose (HG) medium without Phenol Red, supplemented with 10% Fetal Calf Serum (FCS) and 10% Glutamax (Sigma-Aldrich). The cells were maintained at 37° C. and 5% CO₂. At 24-48 hours before treatment, cells were seeded in 6 well plates and allowed to adhere. After adherence and reaching 80% confluence, cells were treated with indicated chemicals (or 0.01% DMSO as vehicle) for 6 h.

For overexpression of GBA2 in HeLa cells, a plasmid containing the cDNA encoding the human GBA2 gene fused to a CMV promoter (pDEST-zeo-hGBA2 (Lelieveld et al, 2020)) was transfected into ˜70% confluent HeLa cells. This plasmid was mixed with FuGENE HD transfection reagent (Promega, Madison, WI, USA) and 500 μl of serum-free DMEM. The mixture was incubated for 20 min at room temperature and added to the HeLa cells cultured in supplemented DMEM. After 2 days, the medium was changed, and chemical treatments were performed, as indicated, for 6 h.

For knockdown of GBA1 and GBA2, lentiviral particles containing the shRNAs for GBA1 and GBA2 were produced. For this purpose we used plasmids developed by The RNAi Consortium containing shRNA against GBA1 (TRCN0000029294) and GBA2 (TRCN0000049585). These plasmids were mixed with a packaging plasmid (psPAX2), and a lentivirus envelope vector (pMD2.G), in 30 μl of transfection reagent (Lipo293D). The mixture was incubated for 20 min at room temperature and added to HEK293T Lenti-X cells cultured in complete DMEM media. After 6 hours, the medium was replaced with complete DMEM containing 25 mM HEPES. After 24 h cells were washed twice with PBS, the medium was replaced with fresh DMEM with HEPES, and at 48 and at 72 h lentivirus particles were harvested. The media from the flasks were collected into 50 ml sterile falcon tubes and centrifuged at 3,000 rpm for 7 min. The supernatants were filtered through a 0.45 m filter. A titration was performed to find the optimal amount of virus needed for efficient knockdown. Based on these experiments, aliquots (2 ml) of lentivirus were added to 3 ml serum-free DMEM containing 10 g/ml polybrene. This mixture was added to Hela cells cultured in fresh serum-supplemented DMEM at 70-80% confluence in T75 flasks. The medium was changed after 12-16 h, and after 48 h, the cells were seeded into 6 well plates. Then, chemical treatments, as indicated, were added for 6 h and cells were collected for qPCR analysis, which was also used to confirm GBA1 and GBA2 knockdown.

### Activity-Based Probe Assay

An activity-based probe (ABP) assay was conducted as previously described by Lelieveld et al., (2019). For experiments in zebrafish, 5 dpf larvae (10 per sample) were amputated and euthanized 4 h after amputation with 0.02% tricaine on ice. Excess water was removed, 50 μL homogenization buffer (25 mM KPI, pH 6.5, 0.1% v/v Triton-X) was added, and samples were homogenized by sonication on ice and stored at −20° C. Additionally, to determine the enzyme activity at the site of inflammation, larvae were transected at 4 h after the amputation into two parts: the part anterior to the yolk extension and the part posterior to the yolk extension, and they were collected separately (30 per sample). For experiments in HeLa cells, cells were grown to 80-90% confluence and received vehicle (0.001% DMSO) or TNF-α treatment for 6 hours. Homogenization buffer (25 mM KPI, pH 6.5, 0.1% v/v Triton-X) was added and the samples were sonicated on ice and stored at −20° C.

A β-glucosidase probe (JJB367-alkyl Cy5) in DMSO was diluted in McIlvaine buffer (citric acid/sodium phosphate dibasic: 100 mM/200 mM mixture, pH 5.8) and 10 μL was added to 10 μl of the protein sample (10 μM final probe concentration in 1% DMSO) and incubated at 37° C. for 30 min. Laemmli sample buffer was added (15 μL) and samples were incubated at 95° C. for 5 min. After that, they were immediately loaded onto a 12% SDS-PAGE gel (Bio-Rad Laboratories). The gels were run at 90V for ˜2 h and analyzed using a Typhoon FLA 9000 Gel Imager (GE Healthcare; Cy5 (λEX: 635 nm, λEM: 665 nm), 750 V, pixel size 100 μm). The intensities of the protein bands were quantified using ImageJ software. In each experiment, four biological replicates were used, except for the experiment in which the larvae were split in an anterior and posterior part. In the latter experiments two biological replicates were used. The total protein concentration of the samples was quantified using a Bradford assay kit (Bio-Rad) to enable normalization for total protein concentrations.

### Enzyme Activity Assay

Zebrafish larvae were amputated at 5 dpf, and were euthanized with 0.02% tricaine on ice at 4 h post amputation and collected in Eppendorf tubes (10 larvae per sample). All excess water was removed, 50 μL homogenization buffer (25 mM KPI, pH 6.5, 0.1% Triton-X) was added, and samples were homogenized by sonication on ice and stored at −20° C. Of the homogenates, 12.5 μL (the equivalent of approximately 1 larva) was added to a black flat bottom 96 well plate on ice along with 12.5 μL Milli-Q water containing 5% DMSO (with and without GBA1 or GBA2 inhibitors). The GBA1 inhibitor (ME656) solution was prepared at a final concentration of 1 μM. The GBA2 inhibitor (MZ31) was used at a final concentration of 100 nM. The plate was incubated at 37° C. for 30 min to allow inhibitors to bind to the enzyme. A volume of 100 μL of 4MU (4-methylumbelliferyl-β-D-glucopyranoside) in 150 mM McIlvaine buffer (pH 5.8) was added. The blank contained 12.5 μL of homogenate, 12.5 μL of Milli-Q with 5% DMSO, and 100 μL of 150 mM McIlvaine buffer (pH 5.8). The plate was, after brief shaking, incubated at 37° C. for 2 h. Following the incubation period, the plate was placed on ice and 200 μL STOP buffer (1 M NaOH-glycine, pH 10.3) was added to each well. After stopping the reactions, a 4MU standard was made (10 μL of known 4MU standard (1 nmol), 15 μL KPI buffer, 100 μL 150 mM McIlvaine (pH 5.8), and 200 μL STOP buffer) and added to a separate well. Subsequently, a blank for the 4MU standard was prepared (10 μL of Milli-Q, 15 μL KPI buffer, 100 μL 150 mM McIlvaine (pH 5.8), and 200 μL STOP buffer) and added to a separate well. The fluorescence intensity in all wells was then measured using a fluorescence spectrometer LS-55 (PerkinElmer). Three biological replicates were used. Specific activity was calculated for each condition using the equation below (Kallemeijn et al., (2017). Investigations on therapeutic glucocerebrosidases through paired detection with fluorescent activity-based probes. PLoS One 12, e0170268):

$$Specific activity in \frac{\frac{π mol}{h}}{mL} = \frac{( Arbs X - Arbs blank )}{Arbs 4 MU std} * ( \frac{60}{minutes} ) * ( \frac{1000}{μ L} )$$

Where Arbs X is the absorbance of the sample, Arbs blank is the absorbance of the blank, Arbs 4MU std is the absorbance of the 4MU standard. To be able to normalize the obtained enzymatic activities per mg of protein, total protein concentration was measured using the Pierce™ BCA Protein Assay Kit (Fisher Scientific) according to the manufacturer's instructions.

### Competitor Binding Assay

To determine the relative GR binding affinities of different compounds, the PolarScreen™ Glucocorticoid Receptor Competitor Assay Kit (ThermoFisher) was used following the manufacturer's instructions. Briefly, test compounds were first dissolved in DMSO to a 10 mM (100×) stock concentration and diluted further in GR buffer (100 mM potassium phosphate (pH 7.4), 200 mM Na2MoO4, 1 mM EDTA, and 20% DMSO). Serial dilutions of the compounds were transferred to a Corning® black 384-well plate and Fluormone GS Red™ was added, followed by GR Full Length (partially purified receptor in storage buffer). The plate was incubated for 4 h at RT in the dark and fluorescence polarization was measured with a CLARIOStar Microplate Reader (BMG Labtech). Obtained values were normalized to the assay maximum (no ligand) and minimum control (10 μM dexamethasone), and relative percentages of polarization were determined. Dose-response curves were fitted enabling the calculations of IC50s values for each compound. Experiments were performed in duplicate.

### Induced Arthritis Assay

The effects of a compound in treating inflammatory disease can be investigated using the collagen antibody-induced arthritis (CAIA) model in mouse. While the method has been described below for the compound GbPdn, the skilled person will readily appreciate how to generalise this for any relevant compound.

Male BALB/cAnNCrl mice (±8 weeks of age) were purchased from Charles River. Four or five mice were housed per cage under specific pathogen-free (SPF) conditions in individually-ventilated cages. The experiment was reviewed by the Animal Welfare Body of the LUMC and executed under a license granted by the Central Authority for Scientific Procedures on Animals under license and study numbers AVD11600202115391/PE.15391.01.002, in accordance with the Dutch Act on Animal Experimentation and EU Directive 2010/63/EU.

All mice received 1.5 mg of a CAIA-cocktail (Chondrex) intravenously on day 1, followed by intraperitoneal (i.p.) injection of 50 μg LPS (Chondrex) on day 4. Mice were randomly divided into six groups. Each group contained four mice, except the 25 mg/kg Pdn and GbPdn groups, which had five mice per group. Pdn and GbPdn were diluted in 10% EtOH (Vehicle), and administered via i.p. injection at doses of 12.5 and 25 mg/kg for Pdn, and 12.5, 25, 50 mg/kg for GbPdn, daily from day 7 until day 15. Arthritis was scored daily from day 5 until day 15 as described in Jansen et al. (Jansen D T et al. Abatacept decreases disease activity in a absence of CD4(+) T cells in a collagen-induced arthritis model. Arthritis Res Ther 2015; 17: 220). Scores were assigned blinded. Blood was collected by tail vein sampling at day 0 (75 μl), 10 (40 μl), 14 (40 μl), and 15 (75 μl). The blood collection on day 14 was performed after 6 h fasting. On day 15, mice were sacrificed using carbon dioxide, and tissue samples were collected that were immediately frozen in liquid nitrogen. Applied humane endpoints were a total body weight loss of >20% in comparison to the day of LPS treatment or reaching an arthritis score of 45.

IL-6 concentrations were determined by ELISA in the serum collected at days 10 and 15, using the IL-6 mouse ELISA kit (Invitrogen), following the manufacturer's protocol. The insulin concentration was determined in serum collected at day 14, using the Ultra-sensitive mouse insulin ELISA kit (Crystal Chem), following the manufacturer's protocol. Corticosterone concentrations were determined in serum collected at day 15, using the Corticosterone HS (high sensitivity) enzyme immunoassay kit (Immunodiagnostic Systems), following the manufacturer's protocol. Expression levels of various GR target genes were determined by qPCR in the tissue samples (homogenized in TRIzol Reagent (ThermoFisher)) collected at day 15.

### Liquid Chromatography/Mass Spectrometry Cleavage Confirmation Assay

The ability of GBA2 to cleave a saccharide from a compound can be assessed with the following assay, which is performed in zebrafish, with liquid chromatography/mass spectrometry used to assess the resulting products. In the method below, the compound GPdn is used, together with appropriate standards of Pdn and GPdn. This method can be adapted to other glycosylsated compounds, by selection of appropriate standards for preparing the LC/MS calibration curves.

One hundred wild type zebrafish larvae (in the presence or absence of the Gba2 inhibitor MZ31), or gba2−/− larvae, were tail-wounded and treated with GbPdn at 5 dpf for 6 h (2 h pre-wounding and 4 h post-wounding). For the groups treated with MZ31, this compound was added 24 h before wounding. Samples were collected in a cell strainer and quickly washed with MilliQ water three times to remove residual treatment solution. The washed larvae were transferred to 2 ml Eppendorf tubes which were immediately immersed into liquid nitrogen. The larvae were lyophilized overnight before they were homogenized with glass beads (5 mm) at 30 Hz for 2 min. The powdered fish were extracted with 1 ml of 80% methanol by ultrasonication for 20 min. The mixture was filtered through a 0.22 μm RC membrane.

To perform LC/MS, calibration curves were made by using 2.5, 5, 25, 100, 500, 1000, and 5000 μg/ml of prednisolone, 21-glucose prednisolone, and 21-gentiobiose prednisolone, spiked in the blank sample (derived from non-treated larvae). The compounds were analyzed by utilizing an Acquity BEH C18 column (50×2.1 mm, 1.7 μm (Waters, USA)) on a Shimadzu LC-30AD (Japan) hyphenated to a SCIEX Q-Trap 6500+ (Framingham, MA, USA). Separations were performed using three mobile phases: (A) water with 0.1% acetic acid; (B) ACN: methanol (9:1, v/v) with 0.1% acetic acid; (C) Isopropanol with 0.1% acetic acid at 40° C. at a flow rate of 0.5 ml/min with 25% of B and 1% of C in 3 min. Multiple reaction monitoring (MRM) was utilized in MS/MS acquisition in positive electrospray ionization mode with spray voltage 4.5 kV, capillary temperature 600° C., sheath gas 50, auxiliary gas 50, curtain gas 40, and collision energy 20 V. Data were acquired using Sciex Analyst software (Version 1.7, Framingham, MA, USA) and peak integration used Sciex OS (Version 1.4.0, Framingham, MA, USA).

### EXAMPLES

In the synthesis examples (Examples 1 and 2), all reagents employed were of American Chemical Society (ACS) grade of higher and were used without further purification unless otherwise stated. The NMR characterization was obtained using a Bruker 500 (500 MHz) using DMSO-d6. HRMS analyses were performed on a Shimadzu Nexera X2 UHPLC system with a Waters Acquity HSS C18 column (2.1×100 mm, 1.8 μm) at 30° C. and equipped with a diode array detector. The following solvent system, at a flow rate of 0.5 mL/min, was used: solvent A, 0.1% formic acid in water; solvent B, 0.1% formic acid in acetonitrile. Gradient elution was as follows: 95:5 (A/B) for 1 min, 95:5 to 15:85 (A/B) over 6 min, 15:85 to 0:100 (A/B) over 1 min, 0:100 (A/B) for 3 min, then reversion back to 95:5 (A/B) for 3 min. This system was connected to a Shimadzu 9030 QTOF mass spectrometer (ESI ionisation) calibrated internally with Agilent's API-TOF reference mass solution kit (5.0 mM purine, 100.0 mM ammonium trifluoroacetate and 2.5 mM hexakis(1H,1H,3H-tetrafluoropropoxy)phosphazine) diluted to achieve a mass count of 10000. Preparative HPLC runs were performed on a BESTA-Technik system with a Dr. Maisch Reprosil Gold 120 C18 column (25×250 mm, 10 μm) and equipped with a ECOM Flash UV detector monitoring at X nm. The following solvent system, at a flow rate of 12 mL/min, was used: solvent A, 0.1% TFA in water/acetonitrile 95/5; solvent B, 0.1% TFA in water/acetonitrile 5/95. Gradient elution was as follows: 95:5 (A/B) for 2 min, 95:5 to 0:100 (A/B) over 55 min, 0:100 (A/B) for 2 min, then reversion back to 95:5 (A/B) over 1 min, 95:5 (A/B) for 2 min.

While the examples and reaction schemes provided illustrate the synthesis of specific glycosylated corticosteroids, as the skilled person will appreciate, these methods may be generalised to synthesise other glycosylated corticosteroids, for example with different saccharides and/or with different corticosteroids.

### Example 1: Synthesis of 21-O-Glucose-Prednisolone

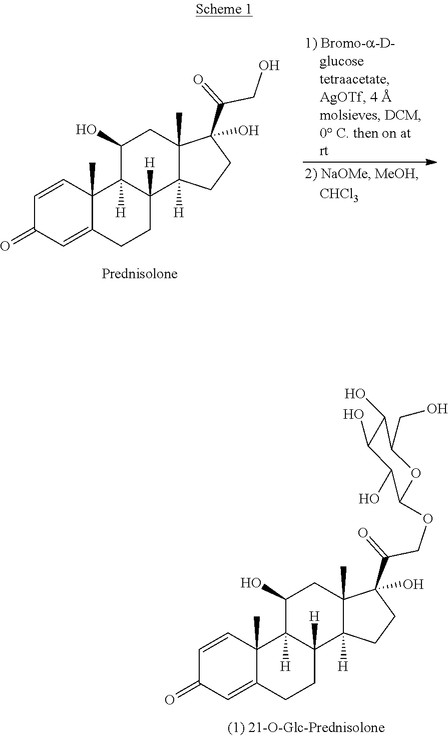

21-O-Glucose-Prednisolone was prepared according to reaction scheme 1. Under an argon atmosphere, prednisolone (0.50 g, 1.39 mmol), silver triflate (1.1 eq, 0.39 g, 1.53 mmol), ground 4 Å molecular sieves (1.2 g) were stirred in 10 ml of DCM at room temperature for 1 h. The reaction mixture was cooled to 0° C. and bromo-a-D-glucose tetraacetate (1.1 eq, 0.63 g, 1.53 mmol) in 10 ml of DCM was added dropwise over 1 h. After stirring the reaction mixture for 1 h at 0° C., the ice water bath was removed and the mixture was stirred over night at room temperature. The next day the reaction mixture was filtered over Celite and a saturated solution of sodium bicarbonate was added to the filtrate. After 2.5 h the organic layer and water layer were separated. The organic layer was then washed with brine, dried over sodium sulfate and concentrated in vacuo. The mixture was then applied to a silica column and eluted with a gradient of 100% DCM to 70:30 DCM/acetone to provide the intermediate product in a partially purified state deemed suitable for the subsequent deprotection step. The peracetylated glucose-prednisolone conjugate was then dissolved in 3 ml of anhydrous methanol and 1.5 ml of anhydrous chloroform and 0.32 ml of 25% wt sodium methoxide in methanol was added dropwise under an argon atmosphere. After stirring for 1 h, Dowex 50WX8 (H+) resin was added to neutralize the mixture. The resin was then removed by filtration and the filtrate concentrated in vacuo. The residue was purified using RP-HPLC and lyophilized to obtain 125 mg of 21-O-Glc-prednisolone as a fluffy white powder in 17% yield over 2 steps. [M+H]+ calculated for C27H38O10, 523,2538, found (HRMS) 523,2538.

¹H NMR (500 MHz, DMSO) δ 7.32 (d, J=10.1 Hz, 1H), 6.16 (dd, J=10.1, 1.9 Hz, 1H), 5.91 (t, J=1.6 Hz, 1H), 5.28 (s, 1H), 4.78 (d, J=18.2 Hz, 1H), 4.59 (s, 1H), 4.34 (d, J=18.2 Hz, 1H), 4.30-4.23 (m, 1H), 4.17 (d, J=7.8 Hz, 1H), 3.69 (dd, J=11.8, 2.0 Hz, 2H), 3.49-3.37 (m, 1H), 3.16-3.07 (m, 2H), 3.06-2.97 (m, 2H), 2.58-2.51 (m, 2H), 2.33-2.22 (m, 1H), 2.11-1.94 (m, 3H), 1.87 (dd, J=13.7, 3.6 Hz, 1H), 1.70-1.57 (m, 4H), 1.44-1.35 (m, 5H), 1.34-1.22 (m, 1H), 1.01 (qd, J=13.2, 4.6 Hz, 1H), 0.89 (dd, J=11.0, 3.5 Hz, 1H), 0.78 (s, 3H).

¹³C NMR (126 MHz, DMSO) δ 208.53, 185.70, 171.05, 157.26, 127.61, 121.72, 102.41, 89.25, 77.53, 77.19, 73.85, 71.69, 70.53, 69.03, 61.61, 55.85, 51.58, 47.19, 44.31, 40.56, 40.39, 40.23, 40.06, 39.89, 39.73, 39.56, 39.43, 34.50, 33.24, 31.86, 31.51, 24.08, 21.37, 17.45.

### Example 2: Synthesis of 21-O-Gentobiose-Prednisolone

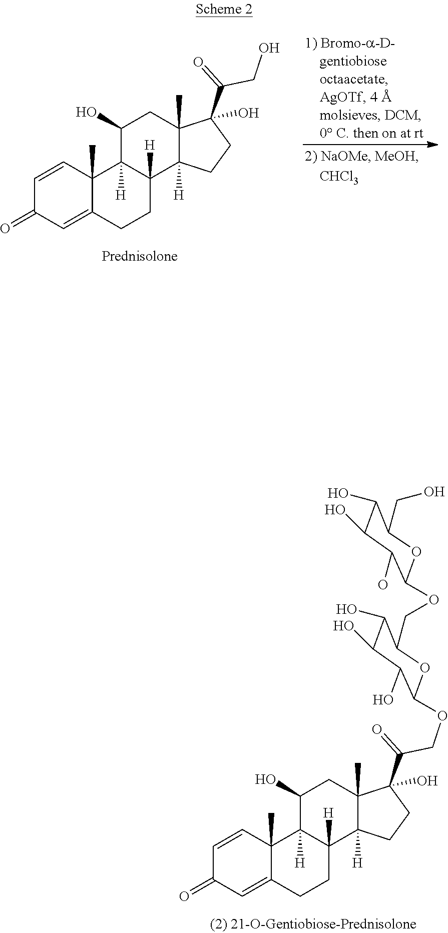

21-O-Gentobiose-Prednisolone was prepared according to reaction scheme 2. Under an argon atmosphere, prednisolone (0.50 g, 1.39 mmol), silver triflate (1.1 eq, 0.39 g, 1.53 mmol), ground 4 Å molecular sieves (1.2 g) were stirred in 10 ml of DCM at room temperature for 1 h. The reaction mixture was cooled to 0° C. and bromo-a-D-gentiobiose octaacetate (1.1 eq, 1.10 g, 1.53 mmol) in 10 ml of DCM was added dropwise over 1 h. After stirring the reaction mixture for 1 h at 0° C., the ice water bath was removed and the mixture was stirred over night at room temperature. The next day the reaction mixture was filtered over Celite and a saturated solution of sodium bicarbonate was added to the filtrate. After 2.5 h the organic layer and water layer were separated. The organic layer was then washed with brine, dried over sodium sulfate and concentrated in vacuo. The mixture was then applied to a silica column and eluted with a gradient of 100% DCM to 70:30 DCM/acetone to provide the intermediate product in a partially purified state deemed suitable for the subsequent deprotection step. The peracetylated gentiobiose-prednisolone conjugate was then dissolved in 3 ml of anhydrous methanol and 1.5 ml of anhydrous chloroform and 0.32 ml of 25% wt sodium methoxide in methanol was added dropwise under an argon atmosphere. After stirring for 1 h, Dowex 50WX8 (H+) resin was added to neutralize the mixture. The resin was then removed by filtration and the filtrate concentrated in vacuo. The residue was purified using RP-HPLC and lyophilized to obtain 221 mg of 21-O-gentiobiose-prednisolone as a fluffy white powder in 23% yield over 2 steps. [M+H]⁺ calculated for C₃₃H₄₈O₁₅, 685,3066, found (HRMS) 685,3067.

¹H NMR (500 MHz, DMSO) δ 7.33 (d, J=10.1 Hz, 1H), 6.15 (dd, J=10.0, 1.9 Hz, 1H), 5.91 (t, 1H), 5.24 (s, 1H), 4.83 (d, J=18.3 Hz, 1H), 4.61 (s, 1H), 4.33 (s, 1H), 4.29 (s, 1H), 4.19 (dd, J=10.1, 7.8 Hz, 2H), 3.96 (dd, J=11.5, 1.9 Hz, 1H), 3.66 (dd, J=11.8, 1.7 Hz, 1H), 3.58 (dd, J=11.4, 5.9 Hz, 1H), 3.44 (dd, J=11.6, 5.2 Hz, 1H), 3.34-3.27 (m, 1H), 3.18-3.10 (m, 3H), 3.08-3.01 (m, 3H), 2.99-2.94 (m, 1H), 2.56-2.51 (m, 2H), 2.32-2.25 (m, 1H), 2.07-1.97 (m, 2H), 1.86 (dd, J=13.8, 3.7 Hz, 1H), 1.73-1.52 (m, 2H), 1.45-1.33 (m, 4H), 1.28 (dd, J=11.4, 6.3 Hz, 1H), 0.99 (qd, J=13.1, 4.6 Hz, 1H), 0.88 (m, 1H), 0.78 (s, 3H).

¹³C NMR (126 MHz, DMSO) δ 208.54, 185.75, 171.12, 157.37, 127.58, 122.09, 103.86, 102.78, 89.19, 77.39, 77.22, 77.07, 76.08, 74.00, 73.74, 72.27, 70.56, 70.12, 68.97, 68.74, 61.53, 55.85, 47.28, 47.25, 44.32, 40.51, 40.34, 40.17, 40.00, 39.84, 39.67, 39.50, 39.45, 34.50, 33.36, 31.87, 31.48, 24.05, 21.38, 17.45.

### Example 3: Anti-Inflammatory Action of Ginsenosides

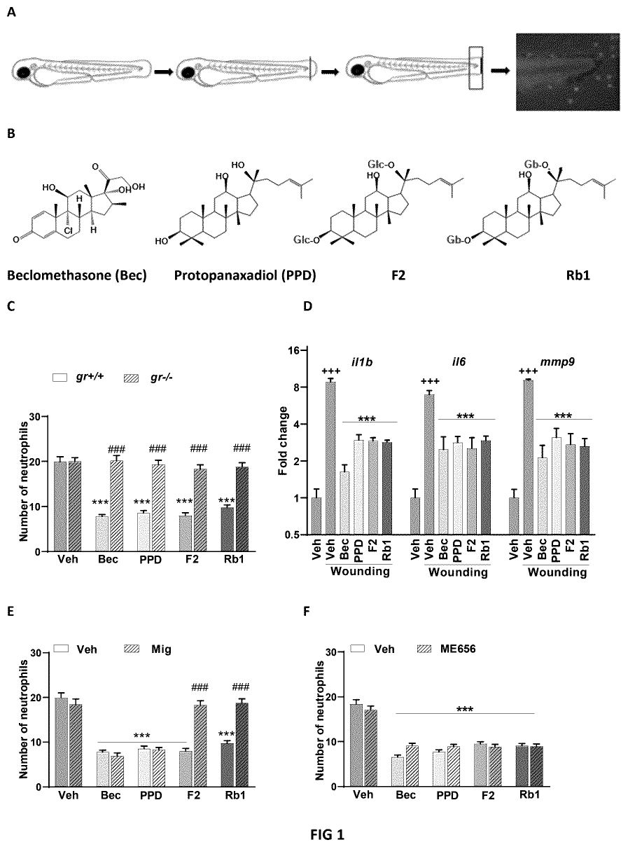

It has previously been reported that ginsenosides have anti-inflammatory effects through activation of the GR (He et al., 2020, Ginsenoside Rg1 Acts as a Selective Glucocorticoid Receptor Agonist with Anti-Inflammatory Action without Affecting Tissue Regeneration in Zebrafish Larvae. Cells 9). In order to study this effect in more detail, zebrafish larvae were induced a local inflammatory reaction by wounding of the tail fin (FIG. 1A), which results in migration of leukocytes towards the wounded area and increased expression of pro-inflammatory genes (Renshaw et al., 2006, Blood 108, 3976-3978; Xie et al., 2019, Dis Model Mech 12). Using this model, it was first investigated whether the anti-inflammatory effects of ginsenosides are dependent on their glycosidation pattern. For this purpose, the ginsenosides Protopanaxadiol (PPD), F2 and Rb1 were used. PPD is not glycosidated and F2 and Rb1 consist of the aglycone PPD with two glycone groups at the C-3 and C-20 positions of the steroid backbone. In F2 these glycones are formed by the monosaccharide glucose, and in Rb1 by the disaccharide gentiobiose which is composed of two glucose molecules joined with a β(1->6) linkage. As a positive control, the synthetic for the anti-inflammatory glucocorticoid drug beclomethasone (Bec) was used. The structures of these compounds are shown in FIG. 1B.

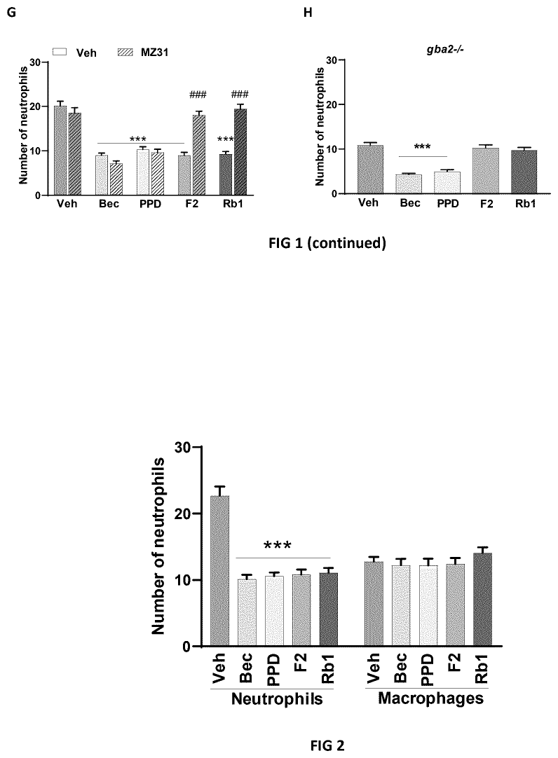

The results show that Bec, as previously demonstrated, decreases the migration of neutrophils towards the wounded area and leaves the migration of macrophages unaffected. The ginsenosides PPD, F2 and Rb1 all show a similar inhibition of the neutrophil migration (FIG. 1C and FIG. 2). As previously shown for the ginsenoside Rg1, this ginsenoside effect is mediated by the Gr, since the inhibition of neutrophil migration was abolished in zebrafish larvae from a mutant line with a deficiency in Gr function (FIG. 1C). Similar results were obtained when we used the expression of pro-inflammatory genes as a readout. Bec, PPD, F2 and Rb1 strongly suppressed the wounding-induced increase in the expression of il1b, il6 and mmp9 (FIG. 1D). Since the effect of the ginsenosides appeared to be independent of the glycosidation pattern, we hypothesized that the glycosidated ginsenosides are metabolized into the aglyconic form. A class of enzymes that can catalyze the hydrolysis of the β-glycosidic bonds between the PPD and the glycone groups are β-glucosidases, and two isoforms occur in vertebrates. The lysosomal acid β-glucosidase, also called glucocerebrosidase (GBA1), is well studied because deficiency in this enzyme results in the most common lysosomal storage disorder in humans, Gaucher disease, which is characterized by the accumulation of glucosylceramide laden tissue macrophages. In addition, a non-lysosomal glucosylceramidase (GBA2) exists, which is not structurally related to GBA1, is located near the cell surface and differs in enzymatic features like specificity towards artificial substrates and inhibitors.

The role of the Gba1 and Gba2 enzymes in the anti-inflammatory effect of ginsenosides was tested using the non-specific chemical GBA inhibitor miglustat, and specific GBA1 and GBA2 inhibitors, ME656 and MZ31 respectively in zebrafish. In the presence of miglustat and MZ31, the reduction in neutrophil migration by F2 and Rb1 was abolished, whereas these inhibitors did not affect the action of PPD (FIG. 1E, G). However, treatment with ME656 did not alter the effects of any ginsenoside (FIG. 1F), indicating that this effect is Gba2-specific. To confirm the dependency of the glycosidated ginsenosdes on Gba2 action, a Gba2-deficient mutant line was used. In larvae from this line, F2 and Rb1 did not affect the neutrophil migration, whereas PPD did (FIG. 1H), similarly to the results obtained with the GBA2 inhibitor. Taken together, these data suggest that the effect of glycosidated ginsenosides depends on GBA2, and not GBA1, hydrolyzing these compounds into their aglyconic form.

### Example 4: Effect of Glycosidated Ginsenosides on Side Effects Compared to Classical Glucocorticoid Drugs

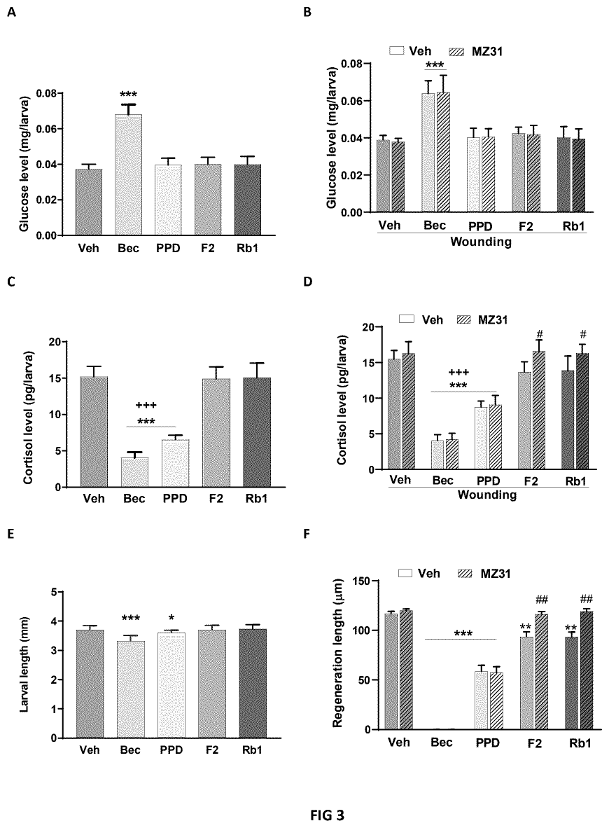

Other ginsenosides were investigated to determine whether a similarly low level of adverse effects and possible side effects are dependent on the glycosidation pattern of the ginsenosides. For this purpose, zebrafish embryos were treated with Bec, PPD, F2 and Rb1, and effects on glucose and cortisol levels, larval length and tissue regeneration upon wounding were monitored, which can be considered proxies for several well-known glucocorticoid side effects observed in the clinic. The anti-inflammatory glucocorticoid drug Bec was used as a positive control, and indeed induced an increase in glucose level (FIG. 3A), and a decrease in cortisol level (FIG. 3C), larval length (FIG. 3E) and tissue regeneration (FIG. 3F, G). The aglyconic ginsenoside PPD did not affect the glucose level (FIG. 3A), but did decrease the cortisol concentration (FIG. 3C), larval length (FIG. 3E) and regeneration (FIG. 3F, G). Interestingly, the glycosidated ginsenosides F2 and Rb1 did not show any effect on glucose either (FIG. 3A), but neither did they affect the cortisol concentration (FIG. 3C), larval length (FIG. 3E) or tissue regeneration (FIG. 3F, G). Upon wounding, the glycosidated ginsenosides Rb1 and F2 did show a minor effect on cortisol and tissue regeneration, which was GBA2-dependent, as demonstrated using the chemical GBA2 inhibitor MZ31 (FIG. 3D,F,H), which could suggest that wounding increases the activity of Gba2, which then hydrolyzes Rb1 and F2.

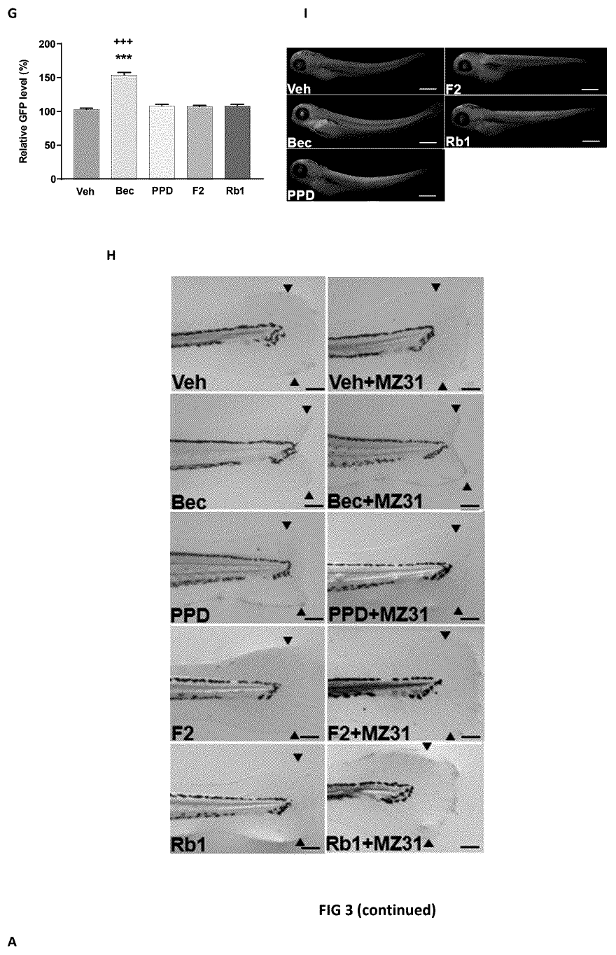

The question that remains is why PPD does not affect the glucose levels, whereas it shows similar (although sometimes smaller) effects as Bec on all other readouts. It is well established that the glucocorticoid-induced increase in glucose concentration solely depends on the transactivation activity of the GR, so we hypothesized that PPD binding to Gr does not induce this transactivation activity. To test this hypothesis, we used a reporter fish line in which the GFP gene is fused to a GRE-containing promoter. Treatment of larvae from this line with Bec resulted in a large increase in GFP signal throughout the body of the larvae, whereas PPD (as well as F2 and Rb1) did not cause an increase in the fluorescence intensity (FIG. 3G,I). Moreover, PPD did not increase the expression of well-known target genes of the transactivation activity of the Gr, fkbp5, pck1 (which plays an important role in the glucocorticoid-induced increase in cortisol concentration), and nfkbiaa (FIG. 4B), confirming that PPD does not induce the transactivation activity of GR.

This demonstrates that the side effects of PPD, which are relatively small because the transactivation activity of Gr is not induced, are abolished upon glycosidation of the aglyconic ginsenoside. Combined with the observed effects of the ginsenosides on the inflammatory response, these results suggest that glycosidation of ginsenosides makes these compounds inactive and that they can be converted into their active, aglyconic form through hydrolysis by the enzyme GBA2, of which the activity is specifically increased in inflamed tissue.

### Example 5: GBA2 Abundance and Activity are Locally Increased at Sites of Inflammation

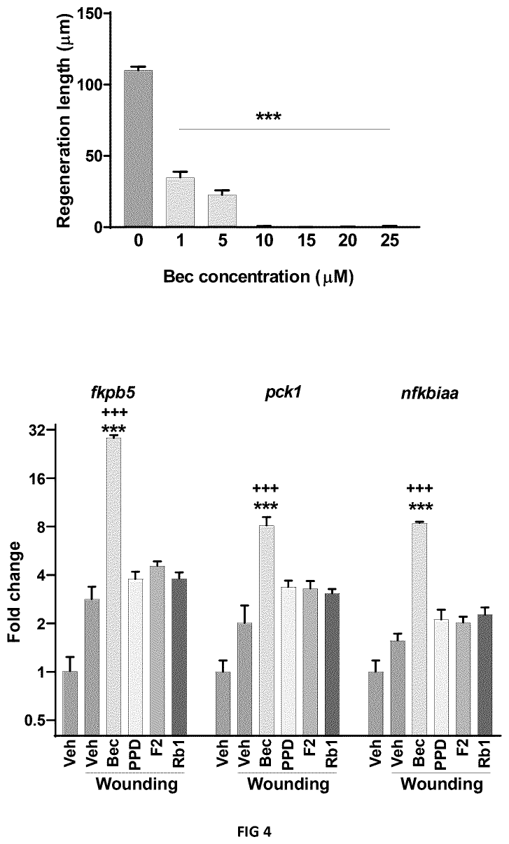

In order to test if the GBA2 activity is increased in inflamed tissue, we wounded the tail fins of zebrafish larvae and measured gba1 and gba2 mRNA concentrations. The results showed that wounding increased gba2 mRNA levels (FIG. 5) in the zebrafish larvae. No effect of wounding was observed on the gba1 mRNA (FIG. 5).

### Example 6: Effect of Glucuronidation of Dexamethasone on Anti-Inflammatory Activity and Side Effects

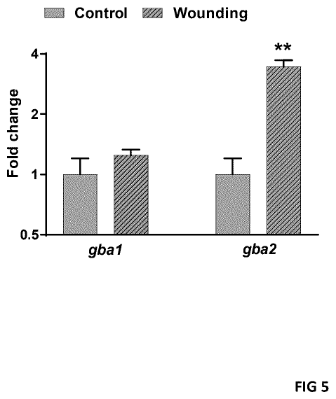

Since glycosidation of the ginsenosides PPD abolished its side effects, we hypothesized that we could develop novel glucocorticoid drugs with reduced side effects by conjugation of glycone groups to synthetic glucocorticoid drugs such as dexamethasone. A glucuronidated form of dexamethasone (i.e. dexamethasone with a glucoronic acid conjugated to the C21 position) form of dexamethasone is commercially available (GDex), in which a glucose group is conjugated at the C-21 position through a β-glycosidic bond. This compound is being developed as a drug against inflammatory bowel disease, based on the hydrolysis of the compound by β-glucosidases from the gut microbiota. First, we determined the anti-inflammatory effects of GDex using our zebrafish tail fin wounding model. The results showed that GDex inhibits neutrophil migration similarly to Bec and Dex, without any effect on macrophage migration (FIG. 6A). This inhibition of neutrophil migration by Bec, Dex and GDex was absent in larvae from the gr mutant line, indicating that the effects of these compounds are mediated by Gr. In addition, the wounding-induced increase in the expression of the pro-inflammatory genes il1b, il6, mmp9 and mmp13 was strongly reduced by GDex, and this effect was similar to the reduction observed after Dex treatment (FIG. 6C). To study possible side effects of GDex, we measured glucose and cortisol levels, and the larval length after treatment with Bec, Dex and GDex. Whereas Dex, like Bec, increased the glucose concentration, and decreased the cortisol level and the length of the larvae, GDex only showed a very minor decrease in cortisol level (FIGS. 6D, E, F). In addition, just like the glycosidated ginsenosides, GDex did not induce the transactivation activity of the Gr, whereas Bec and Dex did, as demonstrated using the GRE:GFP reporter fish line (FIG. 6G) and the expression levels of the Gr genes fkbp5, pck1, and nfkbiaa (FIG. 6H). These data demonstrate that GDex acts like the glycosidated ginsenosides Rb1 and F2, which means it is inactive outside inflamed tissues and is active at these sites of inflammation.

### Example 7: Effect of Glycosidation of Prednisolone on Anti-Inflammatory Activity and Side Effects

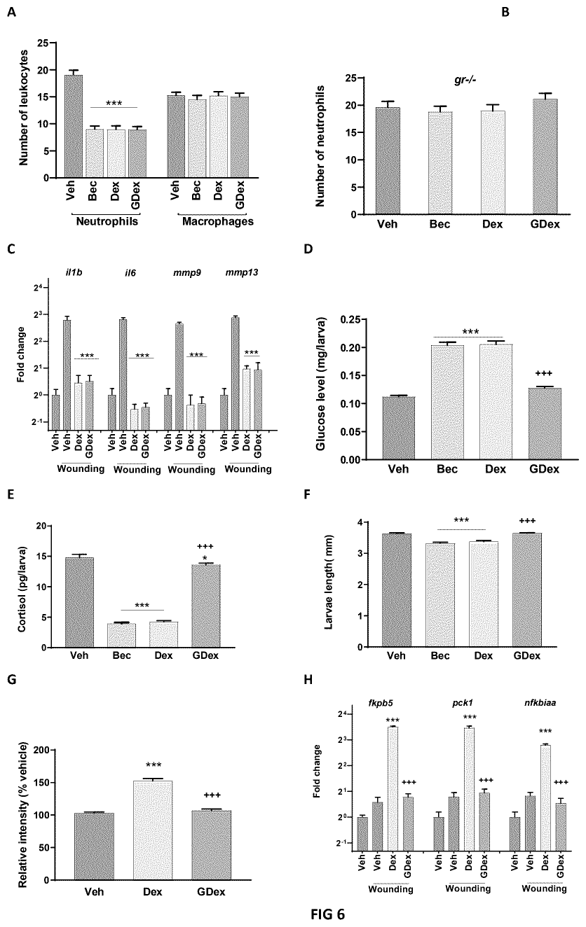

To study whether a similar modification of the glucocorticoid drug prednisolone (Pdn) would yield the same reduction in side effects, we synthesized a glucosidated from of prednisolone (GPdn) in which a glucose group was conjugated at the C-21 position of Pdn. Using our zebrafish tail fin wounding model, we showed that GPdn inhibited the neutrophil migration, similarly to Bec, Dex, GDex and Pdn (FIG. 7A). Like GDex, GPdn did not increase the glucose level (whereas Bec, Dex and Pdn did, FIG. 7B), and only slightly reduced the cortisol concentrations (whereas Bec, Dex and Pdn caused a significantly larger decrease, FIG. 7D). Under wounding conditions, GPdn causes a minor increase in glucose concentration (although this effect is still significantly smaller effect than that of Pdn, FIG. 7C), and a larger decrease in cortisol concentration than in non-wounded larvae (and this effect is also still smaller than the effect of Pdn, FIG. 7E). These additional side effects observed after wounding were reduced upon treatment with MZ31, suggesting that these effects are dependent on the GBA2-mediated conversion of GPdn to Pdn at the wounded site.

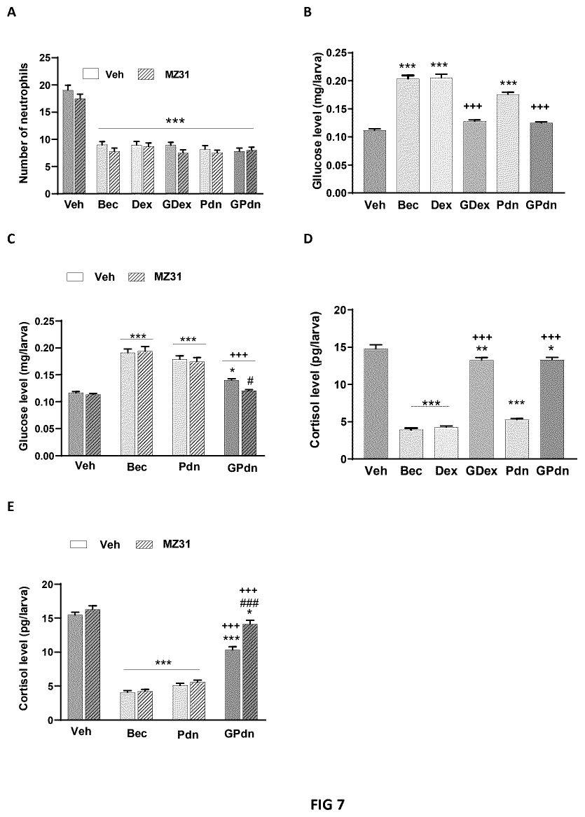

Interestingly, the inhibition of neutrophil migration by GDex and GPdn, unlike the effect of glycosidated ginsenosides, is independent of GBA2 activity, as demonstrated using the GBA2 inhibitor MZ31 (FIG. 7A). This suggests that, like PPD, the uncleaved form is still able to bind and activate Gr sufficiently to decrease the migration of neutrophils. It could therefore be argued that the conjugation of a single monosaccharide was not sufficient to inactivate the glucocorticoid and that larger, more bulky, modifications are necessary. Therefore, we synthesized a second glycosidated form of prednisolone, in which the glycone group conjugated to the C-21 position was formed by the disaccharide gentiobiose. The structures of Pdn, GPdn and this gentiobiosidated Prednisolone (GbPdn) are shown in FIG. 8A. Treatment of zebrafish larvae with GbPdn resulted in a decrease in the migration of neutrophils after tail fin wounding (FIG. 8B) and a suppression of the expression of the pro-inflammatory genes il1b, il6 and il8 (FIG. 8C), and these effects were similar to those observed upon Pdn and GPdn treatment (FIGS. 8B,C). The effect on neutrophil migration of GbPdn, but not GPdn, was abolished by MZ31, suggesting that GbPdn is an inactive molecule and needs removal of the bulky gentiobiose group through hydrolysis by GBA2.

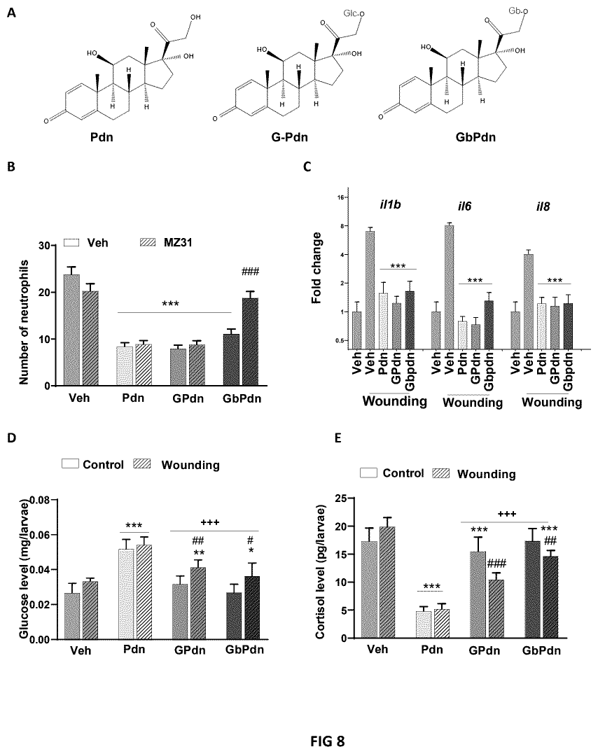

Furthermore, GbPdn did not affect the glucose or cortisol concentration (FIGS. 8B, D) and neither did it affect the length of the larvae (FIG. 9A). A small effect was observed on the regeneration of the tail fin. This effect that was significantly smaller than that of Pdn, and was dependent on Gba2 activity, as demonstrated using MZ31 (FIG. 9C). Under wounding conditions, both GPdn and GbPdn induced an increase in glucose levels (FIG. 8B), and a decrease in cortisol concentration (FIG. 8D), probably as a result of increased GBA2 activity. Using the GRE:GFP reporter zebrafish line, we showed that neither GPdn nor GbPdn induced the transactivation activity of the Gr (FIGS. 9D, E), and this lack of effect was confirmed when we studied the expression of the Gr target genes fkbp5, pck1 and nfkbiaa (FIG. 9F). Apparently, conjugation of a monosaccharide is sufficient to abolish the ability of Pdn to induce the transactivation activity of Gr and abolish almost all glucocorticoid side effects in this model.

### Example 8: Investigating GR Activation in Human Cells In Vitro

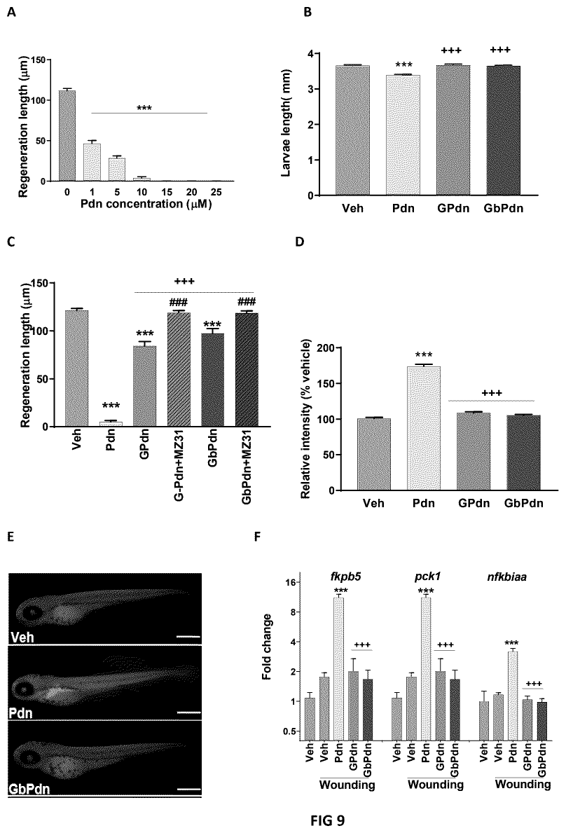

The in vitro binding of Dex, Pdn, GPdn and GbPdn to the human GR was studied using a competitive ligand binding assay. The results showed that Bec and Pdn had similar binding affinities (IC50 of ˜4.8 and ˜12 nM respectively). Pdn had a slightly lower affinity (IC50˜350 nM), but the affinity of GbPdn was dramatically lower (IC50˜17 mM)(FIG. 10). Apparently, the glucosylation of Pdn results in a ˜30 fold decrease in the binding affinity of the ligand, whereas gentiobiosylation decreased the affinity˜1400 times.

### Example 9: Collagen Antibody-Induced Arthritis (CAIA) Mouse Experiment

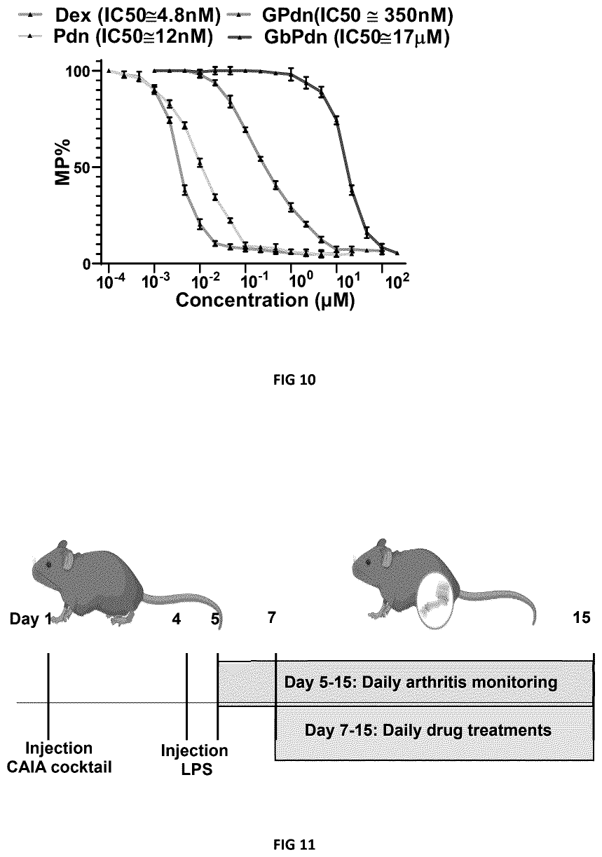

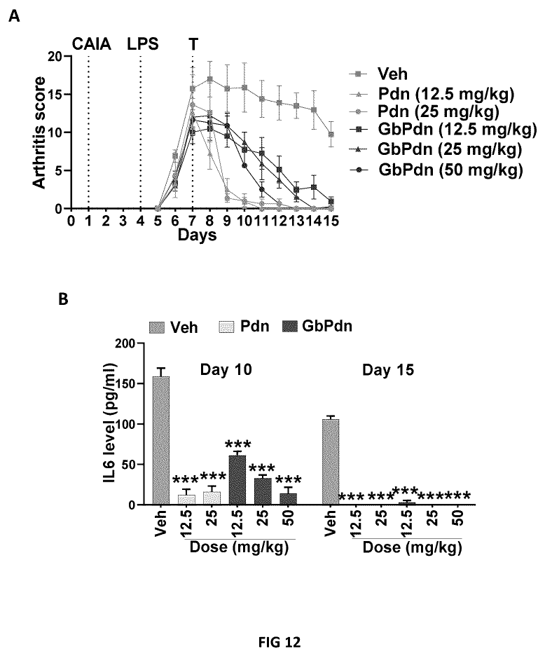

The Induced Arthritis Assay described in the “Assays” section above was performed to investigate the effects of GbPdn in a mammalian model for inflammatory disease. This used the collagen antibody-induced arthritis (CAIA) model in mouse, in which arthritis is induced by injection of a cocktail of antibodies against collagen on day 1 and LPS on day 4 (FIG. 11). Subsequently, we treated the mice daily with vehicle, Pdn (12.5 or 25 mg/kg), or GbPdn (12.5, 25 or 50 mg/kg). Arthritis scores were determined daily from day 5 until 15. Vehicle-treated mice reached high arthritis scores (approximately 15) and these levels remained stable until day 14 (FIG. 12A). In contrast, the scores of Pdn and GbPdn-treated mice groups decreased rapidly after treatment initiation. The groups treated with Pdn showed maximal reduction (scores approximately 0) on day 11 of the experiment, while the groups treated with 12.5, 25, and 50 mg/kg of GbPdn induced a maximal effect on day 15, 14, and 13 respectively (FIG. 12A).

As a second readout for the inflammatory response in these animals, we measured the serum IL-6 level on day 10 and 15. The results of this experiment showed a significant reduction of the IL-6 level in all treatment groups compared to the vehicle group, with the 12.5 mg/kg GbPdn group showing a slightly higher IL-6 concentration compared to the other groups treated with Pdn or GbPdn (FIG. 12B). On day 15, IL-6 levels were undetectable in the serum from all groups treated with Pdn or GbPdn, except in the 12.5 mg/kg GbPdn group, in which a very low level was detected in two (out of four) mice in the 12.5 mg/kg GbPdn group. Taken together, these data show that GbPdn is a highly efficacious anti-inflammatory drug, showing only a delayed activity compared to Pdn.

1. A compound of formula I: 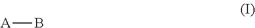
wherein:
A is selected from a monosaccharide and a disaccharide; and
B is selected from an anti-inflammatory moiety or chemotherapeutic moiety;
or a pharmaceutically acceptable salt, stereoisomer, acid, or solvate thereof.

2.-3. (canceled)

4. The compound of claim 1, wherein A is a disaccharide formed from two aldohexoses; optionally wherein A is a disaccharide formed from one or two of each of glucose, galactose, idose, altrose, and any acid form thereof.

5. The compound of claim 1, wherein A is a monosaccharide selected from the group consisting of glucose, galactose, mannose, fructose, and any acid form thereof.

6. The compound of claim 1, wherein B is an anti-inflammatory moiety;
wherein the anti-inflammatory moiety is or comprises a corticosteroid (such as a glucocorticoid), a non-steroidal glucocorticoid receptor agonist, a non-steroidal anti-inflammatory, a disease modifying antirheumatic drug, or rapamycin.

7. (canceled)

8. The compound of claim 1, wherein B is or comprises a corticosteroid (such as a glucocorticoid) selected from the group consisting of a hydrocortisone type corticosteroid, a triamcinolone acetonide type corticosteroid, a methasone type corticosteroid, a betamethasone dipripionate type corticosteroid, and a methylprednisolone aceponate type corticosteroid; or
wherein B is or comprises a molecular radical of alclometasone, amcinonide, beclomethasone, betamethasone, budesonide, chloroprednisone, ciclometasone, ciclesonide, clobetasol, clobetasone, clocortolone, cloprednol, cortifen, cortisol (hydrocortisone), cortisone, cortivazol, cortodoxone, deflazacort, desonide, desoximetasone, dexamethasone, dichlorisone, diflucortolone, diflorasone, difluprednate, formocortal, fluazacort, flucloronide, fludrocortisone, flucortin, flucortolone, flumetasone, flunisolide, fluocinolone, flucinolone acetonide, fluocinonide, fluocortin, fluorometholone, fluperolone, fluprednidene, fluprednisolone, flurandrenolide, fluticasone, halcinonide, halobetasol, halometasone, hydrocortamate, icometasone, isofluprednone, lotoprednol, mazipredone, medrysone, meprednisone, methylprednisolone, mometasone, nicocortonide, paramethasone, prednicarbate, prednisolone, prednimustine, prednisone, procinonide, tixocortol, triamcinolone or triamcinolone acetonide, or any esters thereof.

9.-10. (canceled)

11. The compound of claim 1, wherein B is a chemotherapeutic moiety.

12. A compound of formula II or formula Vb: 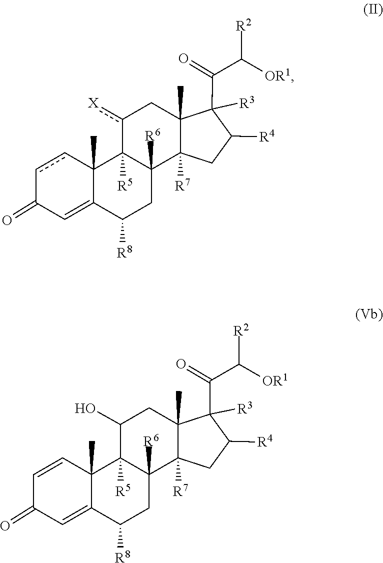
wherein:
R¹ is selected from a monosaccharide or disaccharide;
R² is selected from H, substituted or unsubstituted C₁-C₈ alkyl, substituted or unsubstituted C₁-C₈ alkoxy, substituted or unsubstituted C₃-C₈ alkenyl,
R³ and R⁴ are each independently selected from H, OH, C₁-C₄ substituted or unsubstituted alkyl, OR⁹, OC(O)R⁹; or R³ and R⁴ together with the carbon atoms to which they are attached form a substituted or unsubstituted 5 or 6 membered heterocycloalkyl;
R⁵, R⁶, R⁷ and R⁸ are each independently selected from H, OH, halo, substituted or unsubstituted —C₁-C₄ alkyl, or OR¹⁰;
R⁹ is selected from H, or —C₁-C₈ substituted or unsubstituted alkyl; and
R¹⁰ is selected from H, or —C₁-C₈ substituted or unsubstituted alkyl;
X is selected from ═O or —OH;
or a pharmaceutically acceptable salt, stereoisomer, acid, or solvate thereof.

13.-14. (canceled)

15. The compound of claim 7, wherein R¹ is a disaccharide formed from two aldohexoses, and any acid form thereof, optionally wherein R¹ is a disaccharide formed from one or two of glucose, galactose, idose, and altrose; or
wherein R¹ is gentiobiose.

16. (canceled)

17. The compound of claim 7, wherein R¹ is a monosaccharide selected from the group consisting of glucose, galactose, mannose, fructose, and any acid form thereof.

18. The compound of claim 7, wherein R² is selected from the group consisting of: H, and substituted or unsubstituted C₁-C₈ alkyl.

19. The compound of claim 7, wherein R³ is selected from the group consisting of H, OH, and OC(O)R⁹.

20. The compound of claim 7, wherein R⁹ is a substituted or unsubstituted —C₁-C₈ alkyl.

21. The compound of claim 7, wherein R⁴ is selected from H, OH, or C₁-C₄ alkyl.

22. The compound of claim 7, wherein R³ and R⁴ together with the carbon atoms to which they are attached form a substituted or unsubstituted 5 or 6 membered heterocycloalkyl; or
wherein R³ and R⁴ together with the carbon atoms to which they are attached form a substituted or unsubstituted 1,3 dioxolane.

23.-24. (canceled)

25. The compound of claim 7, wherein R² is H, R³ is OH, and R⁴ is H; or
wherein R² is H, and R³ and R⁴ together with the carbon atoms to which they are attached form a substituted or unsubstituted 1,3 dioxolane; or
wherein R² is H, and R⁴ is C₁-C₄ alkyl: optionally wherein R² is H, and R⁴ is methyl; or
wherein R² is H, R³ is OC(O)R⁹, and R⁴ is H; optionally wherein R⁹ is substituted or unsubstituted —C₁-C₈ alkyl.

26.-28. (canceled)

29. The compound of claim 7, wherein R¹⁰ is H; and/or
wherein R⁵ is selected from H and halo; and/or
wherein R⁸ is selected from H and halo; and/or
wherein R⁶ and R⁷ are each H.

30.-38. (canceled)

39. The compound of claim 7, wherein the compound is a substrate for glucosylceramidase beta 2 (GBA2).

40. The compound of claim 7 selected from: 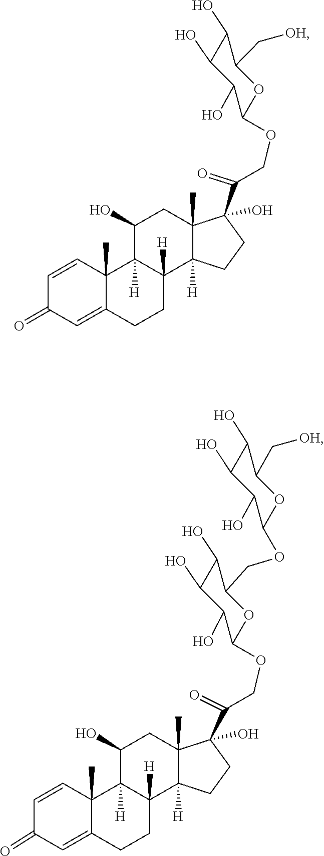
or a pharmaceutically acceptable salt, stereoisomer, acid, or solvate thereof.

41. (canceled)

42. A pharmaceutical composition comprising a compound of claim 7.

43.-44. (canceled)

45. A method for the treatment of inflammation, or an inflammatory condition or other condition characterized by a hyperactivity of the immune system, the method comprising administering an effective amount of the compound of claim 7 to a patient.

46. (canceled)

47. The method of claim 45, wherein the inflammatory or other condition is selected from the group consisting of:
arthrosis, osteoarthritis, gout, rheumatoid arthritis, other auto-immune disorders (such as systemic lupus erythematosus (SLE), inflammatory bowel disease (IBD, including Crohn's disease, ulcerative colitis), multiple sclerosis, chronic inflammatory demyelinating polyneuropathy (CIPD)), infectious disease (such as COVID-19 and tuberculosis), asthma, chronic obstructive pulmonary disease (COPD), allergic and non-allergic rhinitis and sinusitis, tendinitis, nasal polyps, skin conditions (such as rash, dermatitis, itching, eczema, dermatomycosis, lichen (sclerosus or ruber), and psoriasis), ocular disease (such as uveitis, conjunctivitis, macular edema), otitis, thyroiditis, sarcoidosis, myositis, vasculitis, haemorrhoids, organ rejection in transplant recipients and graft versus host disease.

48. (canceled)

49. A method of treating cancer or a condition characterized by hyperproliferation of cells belonging to the immune system, the method comprising administering an effective amount of the compound of claim 7 to a patient.

50.-54. (canceled)
